SEQUENCE LISTING (7 sequences)

SEQ ID NO: 1 - RNA, 41 nt, synthetic construct
  misc_feature 1..41: note = Synthesized DNA
  source 1..41: mol_type = other RNA
taatttctactaagtgtagatgtctggccttaatccatgcc

SEQ ID NO: 2 - DNA, 113 bp, synthetic construct
  misc_feature 1..113: note = Synthesized DNA
  source 1..113: mol_type = other DNA
ctcacgccttatgactgcccttatgtcaccgcttatgtctcccgatatcacacccgttatctcagccctaatctctgcggtttagtctggccttaatccatgcctcatagcta

SEQ ID NO: 3 - RNA, 38 nt, synthetic construct
  misc_feature 1..38: note = Synthesized DNA
  source 1..38: mol_type = other RNA
taatttctactaagtgtagatatcatgcagctcatgcc

SEQ ID NO: 4 - DNA, 24 bp, synthetic construct
  misc_feature 1..24: note = Synthesized DNA
  source 1..24: mol_type = other DNA
tcacctccaccgtgcatttcatca

SEQ ID NO: 5 - DNA, 24 bp, synthetic construct
  misc_feature 1..24: note = Synthesized DNA
  source 1..24: mol_type = other DNA
ttgcgatctgcacacaccagttga

SEQ ID NO: 6 - DNA, 123 bp, Homo sapiens
  source 1..123: mol_type = genomic DNA
tcacctccaccgtgcatttcatcacgcagctcatgcccttcggctgcctcctggactatgtccgggaacacaaagacaatattggctcccagtacctgctcaactggtgtgtgcagatcgcaa

SEQ ID NO: 7 - DNA, 123 bp, Homo sapiens
  source 1..123: mol_type = genomic DNA
tcacctccaccgtgcatttcatcatgcagctcatgcccttcggctgcctcctggactatgtccgggaacacaaagacaatattggctcccagtacctgctcaactggtgtgtgcagatcgcaa

(19) United States
(12) Patent Application Publication
(10) Pub. No.: US 2023/0295689 A1
(43) Pub. Date: Sep. 21, 2023

(54) NUCLEIC ACID DETECTION APPARATUS AND METHOD OF DETECTING NUCLEIC ACID

(71) Applicants: CANON KABUSHIKI KAISHA, Tokyo (JP); CANON MEDICAL SYSTEMS CORPORATION, Tochigi (JP)

(72) Inventors: MASATO MINAMI, Kanagawa (JP); TETSUYA YANO, Ibraki (JP); YOJI YAMAMOTO, Tokyo (JP); MIE OKANO, Kanagawa (JP); SATOSHI OGAWA, Kanagawa (JP); TAKUSHI ICHINOO, Tokyo (JP)

(21) Appl. No.: 18/295,976

(22) Filed: Apr. 5, 2023

Related U.S. Application Data
(63) Continuation of application No. PCT/JP2021/037659, filed on Oct. 12, 2021.

(30) Foreign Application Priority Data
Oct. 13, 2020 (JP) 2020-172561
May 31, 2021 (JP) 2021-091877

(51) Int. Cl. C12Q 1/6806; C12N 9/22; C12N 15/11; G01N 21/64

(52) U.S. Cl. CPC C12Q 1/6806; C12N 9/22; C12N 15/11; G01N 21/6428; G01N 21/6456; C12N 2310/20; G01N 2021/6439

(57) ABSTRACT

Provided is a nucleic acid detection apparatus including: a distribution unit configured to distribute a sample and a detection reagent to a plurality of individual independent separated compartments, the sample containing a target nucleic acid, and the detection reagent containing an effector protein, crRNA to be bound to the target nucleic acid, and a reporter molecule; an activation unit configured to activate the effector protein through binding of the crRNA to the target nucleic acid; a fluorescence generation unit configured to modify the reporter molecule with the activated effector protein to generate fluorescence; a fluorescence detection unit configured to detect the fluorescence; and an identification unit configured to determine, based on a detection result obtained with the fluorescence detection unit, a fluorescence intensity of each of the individual independent separated compartments, and to identify each of the individual independent separated compartments having a fluorescence intensity exceeding a predetermined threshold value.

### CROSS-REFERENCE TO RELATED APPLICATIONS

This application is a Continuation of International Patent Application No. PCT/JP2021/037659, filed Oct. 12, 2021, which claims the benefit of Japanese Patent Application No. 2020-172561, filed Oct. 13, 2020, and Japanese Patent Application No. 2021-091877, filed May 31, 2021, all of which are hereby incorporated by reference herein in their entirety.

### BACKGROUND OF THE INVENTION

### Field of the Invention

The present invention relates to a nucleic acid detection apparatus and a method of detecting a nucleic acid each using a trans-cleavage reaction of a CRISPR-Cas technology and individual independent separated compartments.

### Description of the Related Art

Jennifer Doudna et al. at the University of California have shown that different strains of human papilloma virus (HPV) in a human sample can be accurately detected in distinction from each other through use of Cas12a (Science 27 Apr. 2018: Vol. 360, Issue 6387, pp. 436-439). A complex composed of Cas12a and crRNA specifically recognizes a sequence of target DNA and is bound thereto, and Cas12a cleaves the bound target DNA. In that case, when a reporter molecule in which a fluorescent substance and a quencher are linked to each other by single-stranded DNA is added to a reaction system, Cas12a cleaves the single-stranded DNA of the reporter molecule by a trans-cleavage reaction. Thus, the fluorescent substance and the quencher are separated, and fluorescence is generated. That is, when the target DNA is present in the sample, fluorescence is generated from the fluorescent substance derived from the reporter molecule through activation of the trans-cleavage reaction of Cas12a, and hence the target DNA can be detected based on the fluorescence.

Feng Zhang et al. at the Broad Institute have disclosed a method of detecting target RNA through use of a complex composed of Cas13a and crRNA, and a reporter molecule (Japanese Patent Application Laid-Open No. 2020-501546). The complex composed of Cas13a and crRNA specifically recognizes a sequence of the target RNA and is bound thereto, and Cas13a cleaves the bound target RNA. In that case, when a reporter molecule in which a fluorescent substance and a quencher are linked to each other by RNA is added to a reaction system, Cas13a cleaves the RNA serving as a linking portion to generate fluorescence. Thus, the target RNA can be detected. In addition, in Japanese Patent Application Laid-Open No. 2020-501546, there is a description that a sample containing the target RNA may be distributed to individual independent separated compartments.

When a target nucleic acid having a low concentration is to be detected by one of the methods described in Science 27 Apr. 2018: Vol. 360, Issue 6387, pp. 436-439 and Japanese Patent Application Laid-Open No. 2020-501546, the detection is difficult, or the detection needs to be performed after a step of amplifying the target nucleic acid has been performed. When the method includes the step of amplifying the target nucleic acid, there is a problem in that an operation for amplifying the target nucleic acid is complicated and time-consuming.

### SUMMARY OF THE INVENTION

In view of the foregoing, an object of the present invention is to provide a nucleic acid detection apparatus and method of detecting a nucleic acid, for detecting a target nucleic acid having a low concentration in a simple manner.

According to one aspect of the present invention, there is provided a nucleic acid detection apparatus including: a distribution unit configured to distribute a sample and a detection reagent to a plurality of individual independent separated compartments, the sample containing a target nucleic acid, and the detection reagent containing an effector protein, crRNA to be bound to the target nucleic acid, and a reporter molecule; an activation unit configured to activate the effector protein through binding of the crRNA to the target nucleic acid; a fluorescence generation unit configured to modify the reporter molecule with the activated effector protein to generate fluorescence; a fluorescence detection unit configured to detect the fluorescence; and an identification unit configured to determine, based on a detection result obtained with the fluorescence detection unit, a fluorescence intensity of each of the individual independent separated compartments, and to identify each of the individual independent separated compartments having a fluorescence intensity exceeding a predetermined threshold value.

According to another aspect of the present invention, there is provided a method of detecting a nucleic acid including: a distribution step of distributing a sample and a detection reagent to a plurality of individual independent separated compartments, the sample containing a target nucleic acid, and the detection reagent containing an effector protein, crRNA to be bound to the target nucleic acid, and a reporter molecule; an activation step of activating the effector protein through binding of the crRNA to the target nucleic acid; a fluorescence generation step of modifying the reporter molecule with the activated effector protein to generate fluorescence; a fluorescence detection step of detecting the fluorescence; and an identification step including determining, based on a detection result obtained in the fluorescence detection step, a fluorescence intensity of each of the individual independent separated compartments, and identifying each of the individual independent separated compartments having a fluorescence intensity exceeding a predetermined threshold value.

According to still another aspect of the present invention, there is provided a program for causing a computer included in a nucleic acid detection apparatus to execute the method of detecting a nucleic acid so as to cause the nucleic acid detection apparatus to execute the method.

Further features of the present invention will become apparent from the following description of exemplary embodiments with reference to the attached drawings.

### BRIEF DESCRIPTION OF THE DRAWINGS

FIG. 1 is a functional block diagram of a nucleic acid detection apparatus according to the present invention.

FIG. 2A is a cross-sectional view of a well plate.

FIG. 2B is a cross-sectional view of the well plate with its wells filled with composite particles in which a complex of an effector protein and crRNA is bound to particles.

FIG. 3 is a functional block diagram of another nucleic acid detection apparatus according to the present invention.

FIG. 4 is a block diagram for illustrating a hardware configuration example of the nucleic acid detection apparatus according to the present invention.

FIG. 5 is a flow chart for illustrating a flow of a method of detecting a nucleic acid according to the present invention.

FIG. 6 is a flow chart for illustrating a flow of the method of detecting a nucleic acid in the case of using liquid droplets as individual independent separated compartments.

FIG. 7 is a flow chart for illustrating a flow of the method of detecting a nucleic acid in the case of using wells as individual independent separated compartments.

FIG. 8A is a schematic view for illustrating a fluorescent image of reference liquid droplets.

FIG. 8B is a schematic view for illustrating an image obtained by processing the fluorescent image illustrated in FIG. 8A with image processing software.

FIG. 8C is a schematic view for illustrating a fluorescent image of liquid droplets each containing a sample and a detection reagent.

FIG. 8D is a schematic view for illustrating an image obtained by processing the fluorescent image illustrated in FIG. 8C with image processing software.

FIG. 9A shows a fluorescence microscope image of liquid droplets in Examples.

FIG. 9B shows a fluorescence microscope image of liquid droplets in Examples.

FIG. 9C shows a fluorescence microscope image of liquid droplets in Examples.

FIG. 9D shows a fluorescence microscope image of liquid droplets in Examples.

FIG. 10A shows a fluorescence microscope image of liquid droplets in Examples.

FIG. 10B shows a fluorescence microscope image of liquid droplets in Examples.

FIG. 11A shows a fluorescence microscope image of liquid droplets in Examples.

FIG. 11B shows a fluorescence microscope image of liquid droplets in Examples.

FIG. 12A shows a fluorescence microscope image of liquid droplets in Examples.

FIG. 12B shows a fluorescence microscope image of liquid droplets in Examples.

FIG. 13A shows a fluorescence microscope image of liquid droplets in Examples.

FIG. 13B shows a fluorescence microscope image of liquid droplets in Examples.

FIG. 14A shows a fluorescence microscope image of wells in Examples.

FIG. 14B shows a fluorescence microscope image of wells in Examples.

FIG. 14C shows a fluorescence microscope image of wells in Examples.

FIG. 14D shows a fluorescence microscope image of wells in Examples.

FIG. 15 is a histogram obtained from a fluorescence microscope image of wells in Examples.

FIG. 16A shows a fluorescence microscope image of wells in Examples.

FIG. 16B shows a fluorescence microscope image of wells in Examples.

FIG. 17A shows a bright-field image of wells in Examples.

FIG. 17B shows a fluorescence microscope image of wells in Examples.

FIG. 17C shows a fluorescence microscope image of wells in Examples.

FIG. 18A is a graph showing temporal changes in fluorescence intensity ratios in Examples.

FIG. 18B is a graph showing temporal changes in fluorescence intensity ratios in Examples.

FIG. 18C is a graph showing temporal changes in fluorescence intensity ratios in Examples.

FIG. 19A is a graph showing temporal changes in fluorescence intensity ratios in Examples.

FIG. 19B is a graph showing temporal changes in fluorescence intensity ratios in Examples.

### DESCRIPTION OF THE EMBODIMENTS

Example embodiments of the present invention are now described with reference to the drawings. Like elements or corresponding elements are denoted by the same reference numerals in the drawings, and description thereof may be omitted or simplified.

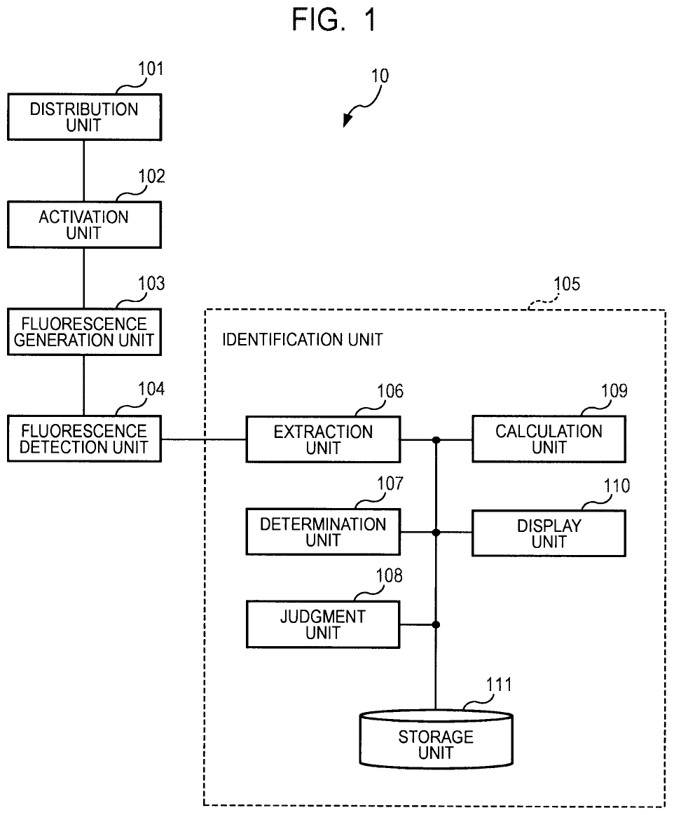

FIG. 1 is a functional block diagram of a nucleic acid detection apparatus 10 according to the present invention. The nucleic acid detection apparatus 10 includes a distribution unit 101, an activation unit 102, a fluorescence generation unit 103, a fluorescence detection unit 104, and an identification unit 105.

The distribution unit 101 distributes a sample containing a target nucleic acid, and a detection reagent to a plurality of individual independent separated compartments. The detection reagent contains an effector protein, crRNA to be bound to the target nucleic acid, and a reporter molecule. In addition, the activation unit 102 activates the effector protein through binding of the crRNA to the target nucleic acid. The fluorescence generation unit 103 modifies the reporter molecule with the activated effector protein to generate fluorescence. The fluorescence detection unit 104 detects the fluorescence. Further, the identification unit 105 determines the fluorescence intensity of each of the individual independent separated compartments, and identifies each individual independent separated compartment having a fluorescence intensity exceeding a predetermined threshold value.

The detection reagent may have the effector protein, the crRNA, and the reporter molecule separate from each other. For example, in the distribution unit 101, before being distributed to the individual independent separated compartments, the sample containing the target nucleic acid may be mixed with the effector protein and the crRNA in advance, followed by further mixing of the reporter molecule therewith.

The identification unit 105 includes an extraction unit 106, a determination unit 107, a judgment unit 108, a calculation unit 109, a display unit 110, and a storage unit 111. The functions of those components included in the identification unit 105 are described later.

Specific configuration examples of the present invention are described below, but the present invention is not limited to the examples described below.

(Effector Protein)

As the effector protein, there may be used, for example, any one of Cas12 or Cas13.

As the Cas12, there may be used, for example, LbCas12a, AsCas12a, FnCas12a, or AaCas12b.

As the Cas13, there may be used, for example, LwaCas13a, LbaCas13a, LbuCas13a, BzoCas13b, PinCas13b, PbuCas13b, AspCas13b, PsmCas13b, RanCas13b, PauCas13b, PsaCas13b, PinCas13b, CcaCas13b, PguCas13b, PspCas13b, PigCas13b, or Pin3Cas13b.

(crRNA)

The crRNA is an RNA designed to contain a base sequence having complementarity to a base sequence possessed by the target nucleic acid. A complex formed of the effector protein and the crRNA specifically binds to the target nucleic acid sequence by virtue of the complementarity possessed by the crRNA.

The crRNA is designed based on the kind of the effector protein to be used and a targeted region in the base sequence possessed by the target nucleic acid.

(Target Nucleic Acid)

Examples of the target nucleic acid may include DNA and RNA. The target nucleic acid may be a nucleic acid that can be applied to diagnosis of a disease state, constitution diagnosis, or the like. Examples of the disease state include cancer, an autoimmune disease, and an infectious disease. Examples of the infectious disease may include infectious diseases caused by DNA viruses, RNA viruses, and the like. Any target nucleic acid may be selected, and the target nucleic acid is not limited to the above-mentioned examples.

(Reporter Molecule)

An example of the reporter molecule may be a molecule in which a fluorescent substance and a quencher are linked to each other by single-stranded DNA, or a molecule in which a fluorescent substance and a quencher are linked to each other by RNA. When Cas12 is used as the effector protein, the reporter molecule is suitably the molecule in which a fluorescent substance and a quencher are linked to each other by single-stranded DNA. For example, DNaseAlert (IDT) may be used as the molecule in which a fluorescent substance and a quencher are linked to each other by single-stranded DNA. In addition, when Cas13 is used as the effector protein, the reporter molecule is suitably the molecule in which a fluorescent substance and a quencher are linked to each other by RNA.

(Amino Compound)

The detection reagent preferably further contains an amino compound. In the presence of the amino compound, the DNA cleavage activity of the effector protein is increased, and hence a time period required for the detection of the target nucleic acid can be shortened.

The detection reagent may have the amino compound separate from the other components, or may have the amino compound in a state of being mixed with any one of the components.

The amino compound is a compound containing an amino group, and in the present invention, any of a primary amine, a secondary amine, and a tertiary amine may be used as the amino compound.

The amino compound is preferably at least one compound selected from the group consisting of: pentaethylenehexamine (the following formula (a1)); spermine (the following formula (a2)); spermine tetrahydrochloride; triethylenetetramine (the following formula (a3)); spermidine (the following formula (a4)); spermidine trihydrochloride; diethylenetriamine (the following formula (a5)); 1,3-diaminopropane (the following formula (a6)); 1,4-diaminobutane (the following formula (a7)); 1,5-diaminopentane (the following formula (a8)); 1,6-diaminohexane (the following formula (a9)); 1,8-diaminooctane (the following formula (a10)); ethylenediamine (the following formula (a11)); ethylamine (the following formula (a12)); propylamine (the following formula (a13)); N,N′-dim ethyl ethylenediamine (the following formula (a14)); N,N-dim ethyl ethylenediamine (the following formula (a15)); N-ethyl ethylenediamine (the following formula (a16)); N-methyl ethylenediamine (the following formula (a17)); and an amino compound having a polyethylene glycol (PEG) structure. Examples of the amino compound having a PEG structure include a compound represented by the following formula (a18), a compound represented by the following formula (a19), and a compound represented by the following formula (a20).

Examples of the compound represented by the following formula (a18) include commercially available Blockmaster (trademark) CE210 (molecular weight of PEG: 2,000, n=45) and Blockmaster (trademark) CE510 (molecular weight of PEG: 5,000, n=114). Examples of the compound represented by the following formula (a19) include SUNBRIGHT (trademark) EA Series. In addition, examples of the compound represented by the following formula (a20) include SUNBRIGHT (trademark) PA Series.

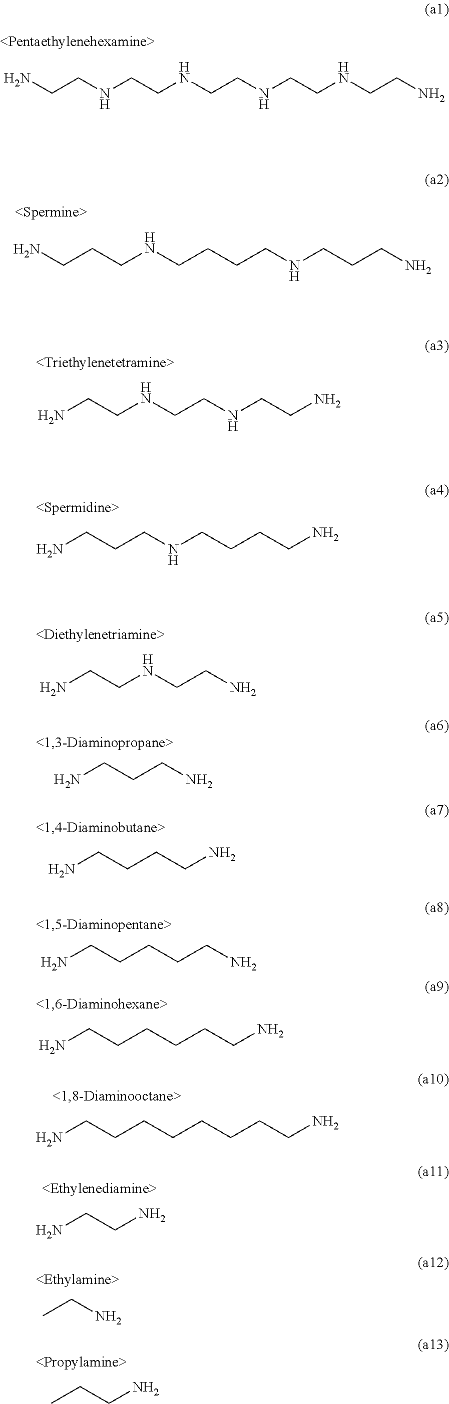

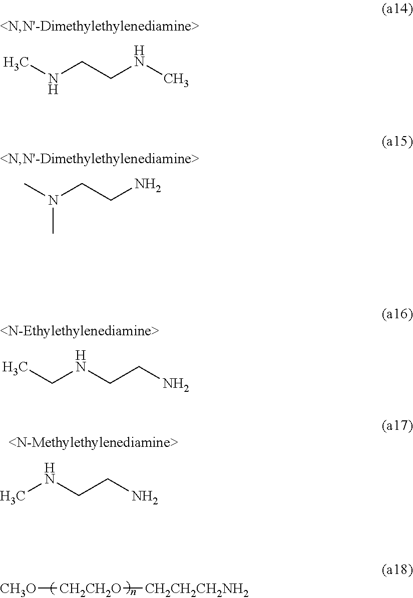

In the formula (a18), “n” represents 30 or more and 1,000 or less.

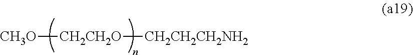

In the formula (a19), “n” represents 30 or more and 1,000 or less.

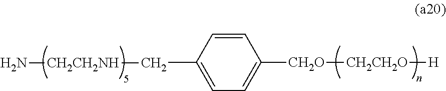

In the formula (a20), “n” represents 30 or more and 120 or less.

The amino compound preferably has —NH₂. In addition, it is more preferred that the amino compound have a plurality of —NH₂'s, or have a plurality of —NH—'s, or have one or more —NH₂'s and one or more —N(CH₃)—'s, or have one or more —NH₂'s and one or more —NH—'s per molecule. In addition, it is still more preferred that the amino compound have one or more —NH₂'s and two or more —NH—'s, or have two or more —NH₂'s and one or more —NH—'s.

(Reaction Buffer)

The detection reagent preferably contains a reaction buffer. The detection reagent may have the reaction buffer separate from the effector protein, the crRNA, the reporter molecule, and the amino compound. In addition, in the detection reagent, at least one selected from the group consisting of: the effector protein; the crRNA; the reporter molecule; and the amino compound may be dispersed in the reaction buffer.

Examples of the reaction buffer include Tris-based buffers and HEPES-based buffers, each of which has proven use in an enzymatic reaction using Cas12a or Cas13a.

Specific examples thereof include Binding buffer (20 mM Tris-HCl (pH 7.6), 100 mM KCl, 5 mM MgCl₂, 1 mM DTT, 5% glycerol, 50 μg/mL heparin), NEBuffer (trademark) 2.1 (10 mM Tris-HCl, 50 mM NaCl, 10 mM MgCl₂, 100 μg/mL BSA, pH 7.9), and FZ buffer (20 mM HEPES, 60 mM NaCl, 6 mM MgCl₂, pH 6.8).

(Particle)

The effector protein is preferably bound to a particle.

The kind of the particle bound to the effector protein is not particularly limited as long as the effector protein can be bound thereto.

The particle may be a primary particle or a secondary particle in which primary particles are aggregated.

The binding between the effector protein and the particle is preferably formed through a reaction utilizing a carboxy group originally possessed by the particle because the effector protein or a linker to be described later can be easily bound. That is, a binding portion between the effector protein and the particle preferably has a structure derived from a carboxy group bonded to the particle.

The effector protein and the particle may be bound to each other via an amide bond. In this case, there is obtained a structure in which C═O derived from a carboxy group contained in the particle and N—H derived from an amino group contained in the protein form the amide bond.

The effector protein and the particle may be bound to each other via a linker. In this case, the “linker” refers to a structure that forms the binding between the effector protein and the particle.

The linker preferably contains a peptide formed of 6 or more and 11 or less consecutive histidine residues.

In this case, the linker may further contain an antibody that binds to the above-mentioned peptide through an antigen-antibody reaction (e.g., an anti-His tag antibody). That is, for example, when an effector protein having the above-mentioned peptide and a particle having the antibody that binds to the peptide are allowed to react to bind the particle and the effector protein to each other, the linker contains the peptide and the antibody. In this case, the particle and the antibody may be bound to each other, for example, via an amide bond formed by allowing a carboxy group originally possessed by the particle and an amino group possessed by the antibody to react with each other. In addition, a commercially available product, for example, EnGen Lba Cas12a (Cpf1) (product name: M0653T, manufactured by NEB Inc., 100 μM) may be used as the effector protein having the above-mentioned peptide.

In addition, the linker may further contain a metal complex that binds to the above-mentioned peptide. That is, the effector protein and the particle may be bound to each other by allowing the effector protein having the above-mentioned peptide and the particle having the metal complex to react with each other. An example of the metal complex that binds to the above-mentioned peptide is a complex of nitrilotriacetic acid or iminodiacetic acid and a divalent nickel ion.

The above-mentioned peptide is particularly preferably a peptide formed of 6 consecutive histidine residues (hereinafter sometimes referred to as “His tag”).

As a binding portion between the effector protein and an effector protein-side linker, there are given: an ε-amino group of a lysine residue possessed by the effector protein; an α-amino group at the N-terminus of the effector protein; various tag peptide sequences or tag proteins artificially inserted into the N-terminus of the effector protein; and the like.

Examples of the tag peptide sequences include a His tag, a HA tag, and a DDDDK tag (FLAG (trademark)). In addition, an example of the tag proteins is Halo-tag (trademark).

As a binding portion between the particle and the linker, various binding portions are given depending on the kind of the linker.

As an example, in the case of binding to the ε-amino group of a lysine residue of the effector protein, or to the α-amino group at the N-terminus of the effector protein, a carboxy group, an aldehyde group, or the like is given as a structure to be utilized for binding to a particle-side linker.

In addition, for example, when an amino group contained in the effector protein and a carboxy group contained in the particle are caused to form an amide bond, a condensation reaction using N-hydroxysuccinimide (NHS)/water-soluble carbodiimide (WSC) may be used.

In addition, as another example, when a His tag artificially inserted into the N-terminus of the effector protein and the particle are bound to each other, an anti-His tag antibody may be used as the linker. In this case, through use of, for example, a carboxy group of the particle as the particle-side linker, the anti-His tag antibody is bound to the surface of the particle through a condensation reaction using an amino group of the anti-His tag antibody and NHS/WSC. As a result, the effector protein can be bound to the particle through use of an antigen-antibody reaction between the His tag at the N-terminus of the effector protein and the anti-His tag antibody.

Other than the anti-His tag antibody, a metal-chelating ligand, such as iminodiacetic acid or nitrilotriacetic acid, is bound, as the linker, to the particle, and a coordinate bond is formed via a metal ion, such as a nickel ion or a cobalt ion. As a result, the effector protein can be immobilized onto the particle.

Other examples of the linker include: any of various tag peptide sequences or tag proteins and a moiety having affinity therefor; a complex of avidin and biotin; and PEG having any of various functional groups at an end thereof. In addition, the binding may be achieved through, for example, physical adsorption between the effector protein and the surface of the particle.

A position in the effector protein to be bound to the particle is preferably such a position that the activity of the effector protein is not inhibited. For example, the effector protein is known to have an active site on the carboxy-terminal (C-terminal) side thereof, and a position distant from the C-terminal side is preferred. For example, the N-terminus is distant from the C-terminal side and can have various tag peptides or tag proteins inserted thereinto, and hence is particularly preferred.

In the present invention, after the effector protein and the crRNA have been bound to each other to form a complex, the effector protein and the particle may be bound to each other to form a composite particle. In addition, after the effector protein and the particle have been bound to each other, the crRNA may be bound to the effector protein to form a composite particle.

As a material for the particle, there are given, for example, a polymer resin (e.g., styrene resin or acrylic resin) particle, a silica particle, a resin particle, an agarose-based resin particle, a metal particle, and a latex particle.

For example, as a commercially available particle that may be used, there are given Magnosphere (trademark) MS300, Magnosphere (trademark) MS160, PureProteome (trademark) Nickel Magnetic Beads, and the like. As the material for the particle, a particle containing a paramagnetic material, a ferromagnetic material, a superparamagnetic material, or the like, such as iron, nickel, and magnetite, is preferably used, but other materials may also be used. When a magnetic particle is used, the position of the effector protein can be easily controlled through application of a magnetic field.

The particle diameter of the particle is preferably 10 nm or more, more preferably 1 μm or more and 10 μm or less.

When the effector protein is bound to a particle, the target nucleic acid can be recovered through use of the properties of the particle. That is, the distribution unit included in the nucleic acid detection apparatus according to the present invention may include a recovery unit, and the recovery unit recovers the target nucleic acid through use of a composite particle formed through binding between the effector protein bound to the particle and the crRNA.

In the recovery unit, after the target nucleic acid has been recovered from the sample, the target nucleic acid may be substantially concentrated by being dispersed in a medium having a smaller volume than the original sample.

For example, with the recovery unit, even a target nucleic acid to be discarded without being caused to fill the individual independent separated compartments can be recovered by allowing the above-mentioned composite particle to act on the target nucleic acid. After the recovery, the target nucleic acid can be detected by filling the individual independent separated compartments with the composite particle that has captured the target nucleic acid. In addition, for example, a trace amount of the target nucleic acid that dissolves in a specimen such as blood or an aqueous solution can be captured on the composite particle and recovered.

In addition, for example, when the sample containing the target nucleic acid is a large-volume solution, the target nucleic acid may be captured by the above-mentioned composite particle before being filled into minute individual independent separated compartments, to fill the composite particle holding the target nucleic acid into the individual independent separated compartments. Thus, the target nucleic acid can be distributed to the individual independent separated compartments with a reduced loss of the target nucleic acid, and the target nucleic acid can be detected with high sensitivity.

In addition, when a magnetic particle is used as the particle, the target nucleic acid can be simply recovered through utilization of magnetism.

(Blocking Agent)

The detection reagent may further contain a blocking agent. That is, at the time of binding the particle and the effector protein to each other, part of the linker binding portion of the particle to which the effector protein has not been bound may be sealed with the blocking agent. For example, when an amino group of the effector protein or the anti-His tag antibody and a carboxy group of the particle are subjected to a condensation reaction through use of NHS/WSC, the particle may have an unreacted carboxy group after the reaction. A carboxy group that has not been bonded to the effector protein may be allowed to react with ethanolamine, PEG having an amino group, or the like serving as the blocking agent.

(Individual Independent Separated Compartments)

The nucleic acid detection apparatus according to the present invention performs a trans-cleavage reaction of a CRISPR-Cas technology in individual independent separated compartments. When the sample containing the target nucleic acid is distributed to the individual independent separated compartments, the sample is apparently brought into a concentrated state, and it is possible to detect the target nucleic acid without performing an amplification step and shorten a time period for fluorescence signal saturation. Further, when the volume per compartment of the individual independent separated compartments is sufficiently reduced, the target nucleic acid included in one compartment can be set to one molecule or less, and when the number of compartments from which fluorescence signals are obtained is counted, it is possible to calculate a concentration of the target nucleic acid in the sample.

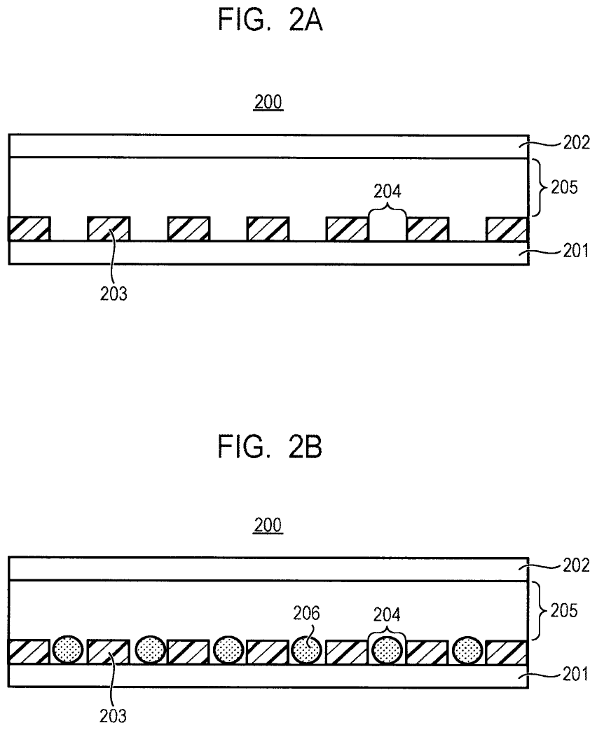

Liquid droplets or wells may be used as the individual independent separated compartments. A water-in-oil type emulsion (W/O emulsion) is preferably used as each of the liquid droplets. In addition, as an example of the wells, it is possible to use wells included in a well plate having such a structure as illustrated in FIG. 2A and FIG. 2B.

FIG. 2A is a cross-sectional view of a well plate 200, and FIG. 2B is a cross-sectional view of the well plate 200 having wells 204 filled with composite particles 206 in which a complex of the effector protein and crRNA is bound to particles. The well plate 200 includes a lower substrate 201, an upper substrate 202, an injection port portion (not shown), and a discharge port portion (not shown), and the lower substrate 201 has hydrophobic partition walls 203 formed thereon. On the lower substrate 201, a plurality of the wells 204 are separated from each other by the partition walls 203.

The lower substrate 201 preferably has a hydrophilic surface, and for example, glass, silicon, or a polymeric resin may be used as a material for the lower substrate 201. In addition, a surface (surface opposed to the lower substrate 201) of the upper substrate 202 is preferably hydrophobic. For example, a hydrophobic resin, a water-repellent resin, or a fluorine-based polymeric resin may be used as a material for the partition walls 203. When the bottom surface of each of the wells 204 is hydrophilic and the top surface of each of the partition walls 203 is hydrophobic, the wells 204 can be efficiently filled with a solution, and in a step of removing an excess of the solution with a hydrophobic solvent, the hydrophobic solvent can be prevented from entering the wells 204.

The wells 204 are recesses for accommodating the solution, and are separated from each other by the partition walls 203. The wells 204 use the lower substrate 201 as their bottom surfaces, and the shape of a region surrounded by the bottom surface and side surfaces of each of the wells 204 may be, for example, a columnar shape or a prismatic shape. In the well plate 200 illustrated in FIG. 2A and FIG. 2B, the depth of each of the wells 204 is equal to the height of each of the partition walls 203.

When the shape of each of the wells 204 is a columnar shape, it is preferred that the diameter of each of the wells 204 be 1 μm or more and 11 μm or less, and the depth of each of the wells 204 be 0.1 μm or more and 10 μm or less. In addition, it is more preferred that the diameter of each of the wells 204 be 1 μm or more and 7 μm or less, and the depth of each of the wells 204 be 1 μm or more and 8 μm or less.

The upper substrate 202 is opposed to the openings of the wells 204 and the top surfaces of the partition walls 203 across a space 205. The space 205 serves as a channel through which various liquids flow, and the various liquids can flow from the injection port portion toward the discharge port portion. That is, after the wells 204 have been filled with the solution, the space 205 is filled with the hydrophobic solvent. When the composite particles 206 in which the complex of the effector protein and the crRNA is bound to particles are used, the wells 204 are filled with the composite particles 206, and the space 205 is filled with the hydrophobic solvent. For example, a fluorine oil or an aliphatic hydrocarbon may be used as the hydrophobic solvent.

The volume of each of the individual independent separated compartments is preferably 0.1 fL or more and 1,000 fL or less, more preferably 0.5 fL or more and 400 fL or less.

When the volume of each of the individual independent separated compartments is 0.1 fL or more, liquid droplets or wells each having such volume can be formed without difficulty. In addition, when the volume of each of the individual independent separated compartments is 1,000 fL or less, the detection time can be made sufficiently short.

(Distribution Unit)

When the individual independent separated compartments are liquid droplets, the distribution unit 101 includes, for example, an emulsification membrane or microchannels. When the distribution unit 101 includes the emulsification membrane, the liquid droplets may be prepared by, for example, using a direct membrane emulsification method or pumping method with a Shirasu porous glass (SPG) membrane of SPG Technology Co., Ltd. or the like. When the distribution unit 101 including the emulsification membrane is used, for example, liquid droplets each having a diameter of from 0.6 μm to 12 μm may be prepared in a combination of Isopar L (aliphatic hydrocarbon, manufactured by Exxon Mobil Corporation) and KF-6038 (surfactant, manufactured by Shin-Etsu Chemical Co., Ltd.).

When the distribution unit 101 includes the microchannels, for example, microchannels of Dolomite Microfluidics may be used as the microchannels. When the distribution unit 101 including the microchannels is used, for example, liquid droplets each having a diameter of from 2 μm to 10 μm may be prepared in each of the following two combinations.
A combination of Isopar L (aliphatic hydrocarbon, manufactured by Exxon Mobil Corporation) and KF-6038 (surfactant, manufactured by Shin-Etsu Chemical Co., Ltd.)
A combination of a mineral oil (aliphatic hydrocarbon) and SPAN-80 (surfactant, manufactured by Tokyo Chemical Industry Co., Ltd.)

When the individual independent separated compartments are wells, the distribution unit 101 includes an injection portion, and the solution is injected into the wells from the injection port portion of the well plate, for example, via a nozzle through use of the injection portion.

In the distribution unit 101, the sample containing the target nucleic acid, and the detection reagent may be mixed in advance before being distributed to the individual independent separated compartments, thus being distributed as a reaction liquid, or may be each individually distributed so as to be mixed with each other in the individual independent separated compartments. The sample containing the target nucleic acid, and the detection reagent are preferably mixed in advance before being distributed to the individual independent separated compartments because a uniformly mixed reaction liquid can be easily obtained.

(Activation Unit)

The activation unit 102 binds the crRNA to the target nucleic acid through appropriate adjustment of an environment such as a temperature in accordance with, for example, the sample, the sequence of the crRNA, and the kind of the effector protein, to thereby activate the effector protein. For example, an incubator may be used as the activation unit 102.

(Fluorescence Generation Unit)

The fluorescence generation unit 103 modifies the reporter molecule with the effector protein through appropriate adjustment of an environment such as a temperature in accordance with, for example, the sample, the kind of the effector protein, and the kind of the reporter molecule, to thereby generate fluorescence. For example, an incubator may be used as the fluorescence generation unit 103.

The activation unit 102 and the fluorescence generation unit 103 may be a single constituent part in the nucleic acid detection apparatus.

(Fluorescence Detection Unit)

The fluorescence detection unit 104 detects the fluorescence generated with the fluorescence generation unit 103. Any device may be used as the fluorescence detection unit 104 as long as the device can detect fluorescence in the individual independent separated compartments, but examples thereof include a plate reader and a fluorescence microscope.

(Identification Unit)

In the identification unit 105, the extraction unit 106 extracts information on the number and relative positional relationship of the plurality of individual independent separated compartments from detection results obtained with the fluorescence detection unit 104. The determination unit 107 determines the fluorescence intensity of each of the individual independent separated compartments identified with the extraction unit 106 based on the detection results obtained with the fluorescence detection unit 104. The judgment unit 108 judges each individual independent separated compartment having a fluorescence intensity exceeding the predetermined threshold value based on the fluorescence intensity determined with the determination unit 107. Thus, each individual independent separated compartment having a fluorescence intensity exceeding the predetermined threshold value is identified.

The identification unit 105 is preferably configured to identify each individual independent separated compartment having a fluorescence intensity exceeding the predetermined threshold value based on a ratio between the fluorescence intensity of a reference compartment and the fluorescence intensity of each individual independent separated compartment. Further, the fluorescence intensity of the reference compartment is preferably a fluorescence intensity acquired using a sample free of the target nucleic acid, and the detection reagent. The fluorescence intensity of the reference compartment may be determined in the same manner as with the individual independent separated compartments except that the reference compartment is free of the target nucleic acid.

The ratio between the fluorescence intensity of the reference compartment and the fluorescence intensity of each individual independent separated compartment is calculated by, for example, using the fluorescence intensity of the reference compartment as a denominator and the fluorescence intensity of each individual independent separated compartment as a numerator. When the ratio between the fluorescence intensity of the reference compartment and the fluorescence intensity of an individual independent separated compartment is less than the predetermined threshold value, the judgment unit 108 deems the fluorescence intensity of that individual independent separated compartment to be equivalent to that of the reference compartment, and judges the individual independent separated compartment to be negative. In addition, when the ratio between the fluorescence intensity of the reference compartment and the fluorescence intensity of an individual independent separated compartment is equal to or more than the predetermined threshold value, the judgment unit 108 judges that individual independent separated compartment to be positive.

The calculation unit 109 calculates the concentration of the target nucleic acid in the sample based on the identified number of individual independent separated compartments each having a fluorescence intensity exceeding the predetermined threshold value. When the concentration of the target nucleic acid in the sample is not calculated, the identification unit 105 does not need to include the calculation unit 109.

The display unit 110 displays information acquired or extracted with the extraction unit 106, the determination unit 107, the judgment unit 108, and the calculation unit 109. The storage unit 111 stores data and the like acquired or extracted with the extraction unit 106, the determination unit 107, the judgment unit 108, and the calculation unit 109.

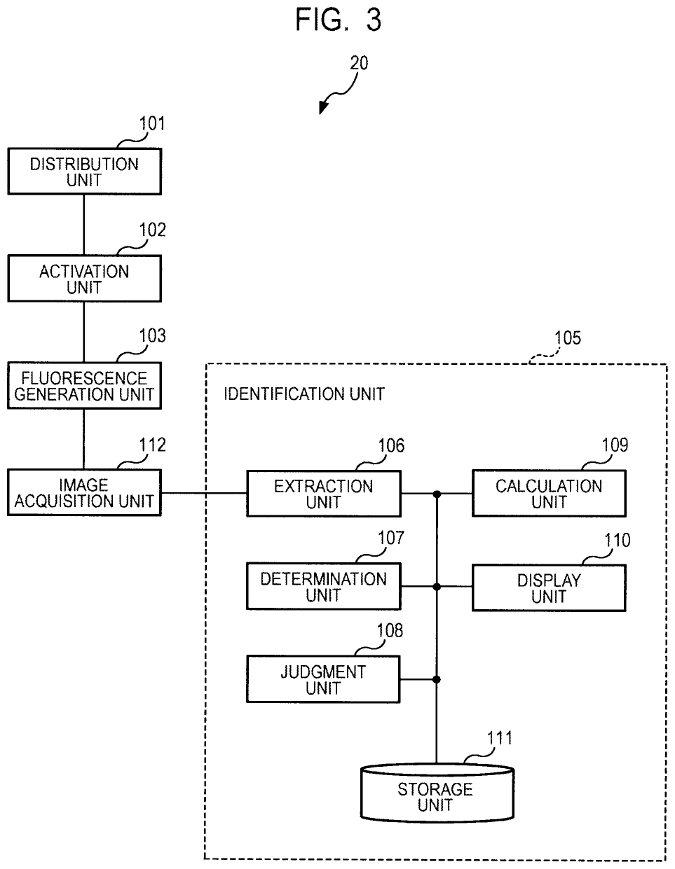

In the nucleic acid detection apparatus according to the present invention, the fluorescence detection unit 104 may be an image acquisition unit. FIG. 3 is a functional block diagram for illustrating a nucleic acid detection apparatus 20 including an image acquisition unit 112 as the fluorescence detection unit 104 in a configuration similar to that of the above-mentioned nucleic acid detection apparatus 10.

The image acquisition unit 112 acquires an image containing the individual independent separated compartments or composite particles in which the complex of the effector protein and the crRNA is bound to particles. The image to be acquired with the image acquisition unit 112 is an image containing the fluorescence generated with the fluorescence generation unit 103 as image information. For example, a fluorescence microscope may be used as the image acquisition unit 112.

In the nucleic acid detection apparatus 20, the extraction unit 106 included in the identification unit 105 preferably extracts information on the number and relative positional relationship of the plurality of individual independent separated compartments based on the image acquired with the image acquisition unit 112. That is, in the nucleic acid detection apparatus 20, the identification unit 105 preferably identifies each individual independent separated compartment having a fluorescence intensity exceeding the predetermined threshold value by processing the image acquired with the image acquisition unit 112.

For example, the extraction unit 106 extracts information on the individual independent separated compartments through use of a region extraction method using brightness information. In particular, when the individual independent separated compartments are liquid droplets, regions corresponding to the liquid droplets on the image each have an outline, and hence the extraction unit 106 may perform processing for extracting the edge of the outline as a closed curve. In addition, the extraction unit 106 may extract the regions corresponding to the liquid droplets on the image by binarizing the image based on brightness information.

Further, the determination unit 107 determines the fluorescence intensity of each of the individual independent separated compartments based on brightness information that each individual independent separated compartment on the image has.

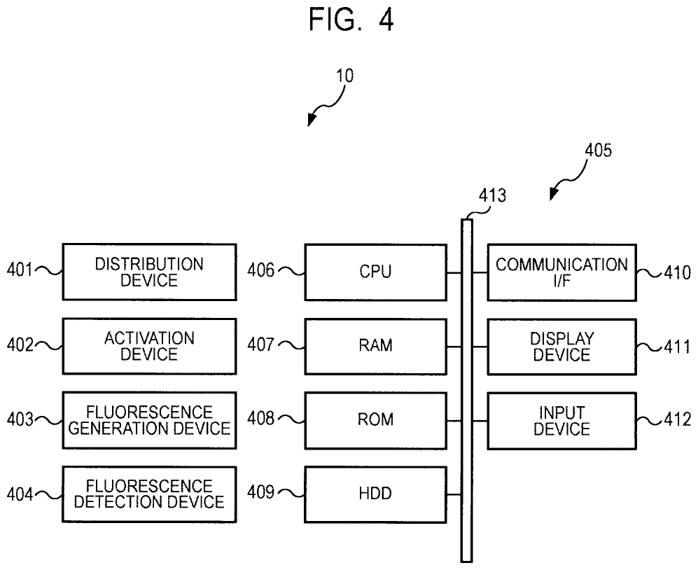

FIG. 4 is a block diagram for illustrating a hardware configuration example of the nucleic acid detection apparatus 10 according to the present invention. The nucleic acid detection apparatus 10 includes a distribution device 401, an activation device 402, a fluorescence generation device 403, a fluorescence detection device 404, and an information processing system 405. The information processing system 405 may be, for example, an individual independent separated compartment identification device.

The distribution device 401, the activation device 402, the fluorescence generation device 403, and the fluorescence detection device 404 are devices configured to execute the functions of the distribution unit 101, the activation unit 102, the fluorescence generation unit 103, and the fluorescence detection unit 104, respectively.

The information processing system 405 has functions of a computer. For example, the information processing system 405 may be configured unitarily with a desktop personal computer (PC), a laptop PC, a tablet PC, a smartphone, or the like. The information processing system 405 has a function of identifying each individual independent separated compartment having a fluorescence intensity exceeding a predetermined threshold value. In addition, the information processing system 405 may further have a function of controlling the operations of the distribution device 401, the activation device 402, the fluorescence generation device 403, and the fluorescence detection device 404 in accordance with a predetermined program.

The information processing system 405 includes, in order to implement functions as a computer that performs arithmetic operation and storage, a central processing unit (CPU) 406, a random-access memory (RAM) 407, a read-only memory (ROM) 408, and a hard disk drive (HDD) 409. The information processing system 405 also includes a communication interface (I/F) 410, a display device 411, and an input device 412. The CPU 406, the RAM 407, the ROM 408, the HDD 409, the communication I/F 410, the display device 411, and the input device 412 are connected to each other via a bus 413. The display device 411 and the input device 412 may be connected to the bus 413 via a drive device (not shown) for driving those devices.

In FIG. 4, the various components forming the information processing system 405 are illustrated as an integrated device, but part of the functions of those components may be implemented by an external device. For example, the display device 411 and the input device 412 may be external devices different from the components implementing the functions of the computer including the CPU 406 and the like.

The CPU 406 performs predetermined operations in accordance with programs stored in, for example, the RAM 407 and the HDD 409, and also has a function of controlling each component of the information processing system 405. The RAM 407 is built from a volatile storage medium, and provides a temporary memory area required for the operations of the CPU 406. The ROM 408 is built from a non-volatile storage medium, and stores required information such as programs to be used for the operations of the information processing system 405. The HDD 409 is a storage device which is built from a non-volatile storage medium, and which stores information on, for example, the number of individual independent separate compartments and positions thereof and fluorescence intensities.

The communication I/F 410 is a communication interface based on a standard, such as Wi-Fi (trademark) or 4G, and is a module for communicating to and from another device. The display device 411 is, for example, a liquid crystal display or an organic light emitting diode (OLED) display, and is used for displaying moving images, still images, characters, and the like. The input device 412 is, for example, a button, a touch panel, a keyboard, or a pointing device, and is used by a user to operate the information processing system 405. The display device 411 and the input device 412 may be integrally formed as a touch panel.

The hardware configuration illustrated in FIG. 4 is an example, and devices other than the illustrated devices may be added, or part of the illustrated devices may be omitted. In addition, part of the devices may be substituted with another device having the same function. Further, part of the functions may be provided by another device via a network, and the functions for implementing the embodiments may be shared and implemented by a plurality of devices. For example, the HDD 409 may be substituted with a solid state drive (SSD) using a semiconductor element such as a flash memory, or may be substituted with cloud storage.

The CPU 406 implements functions of the extraction unit 106, the determination unit 107, the judgment unit 108, and the calculation unit 109 by loading the program stored in the ROM 408 or another place onto the RAM 407 and executing the program. The CPU 406 also implements a function of the display unit 110 by controlling the display device 411. The CPU 406 implements a function of the storage unit 111 as well by controlling the HDD 409.

As a hardware configuration example of the nucleic acid detection apparatus 20 according to the present invention, there is given an example including as the fluorescence detection device 404 an image acquisition device configured to execute the function of the image acquisition unit 112 in the hardware configuration example of the nucleic acid detection apparatus 10 described above. The hardware configuration of the nucleic acid detection apparatus 20 may be the same as that of the nucleic acid detection apparatus 10 except the foregoing.

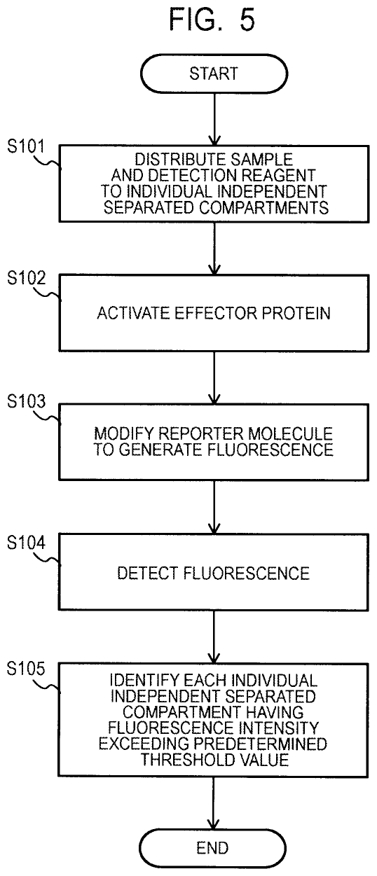

Next, a method of detecting a nucleic acid according to the present invention, which is executed in the nucleic acid detection apparatus described above, is described. FIG. 5 is a flow chart for illustrating a flow of the method of detecting a nucleic acid according to the present invention.

The method of detecting a nucleic acid according to the present invention includes: a distribution step S101 of distributing a sample and a detection reagent to a plurality of individual independent separated compartments, the sample containing a target nucleic acid, and the detection reagent containing an effector protein, crRNA to be bound to the target nucleic acid, and a reporter molecule; an activation step S102 of activating the effector protein through binding of the crRNA to the target nucleic acid; a fluorescence generation step S103 of modifying the reporter molecule with the activated effector protein to generate fluorescence; a fluorescence detection step S104 of detecting the fluorescence; and an identification step S105 including determining the fluorescence intensity of each of the individual independent separated compartments, and identifying each individual independent separated compartment having a fluorescence intensity exceeding a predetermined threshold value.

When the effector protein is bound to a particle, the above-mentioned distribution step may include a recovery step of recovering the target nucleic acid through use of a composite particle formed through binding between the effector protein bound to the particle and the crRNA.

Specific examples of the method of detecting a nucleic acid according to the present invention are described below for a case in which the individual independent separated compartments are liquid droplets and a case in which the individual independent separated compartments are wells.

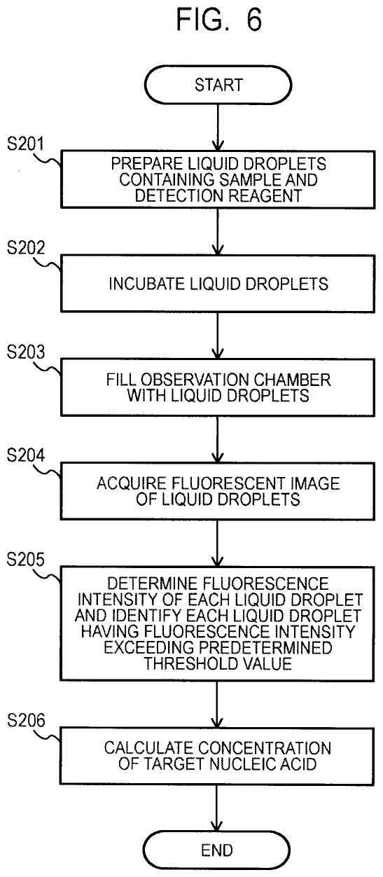

FIG. 6 is a flow chart for illustrating a flow of the method of detecting a nucleic acid according to the present invention in the case where the individual independent separated compartments are liquid droplets.

(Step S201)

Liquid droplets each containing the sample and the detection reagent are prepared. The liquid droplets are each preferably a water-in-oil type emulsion.

(Step S202)

The liquid droplets each containing the sample and the detection reagent are placed in a tube, and are incubated in an incubator at 37° C. However, the reaction temperature may be set to any temperature, and is not limited to 37° C.

Through this incubation, a trans-cleavage reaction of CRISPR-Cas progresses, and fluorescence is generated from a fluorescent substance possessed by the reporter molecule.

(Step S203)

The incubation is ended after a reaction time set in advance, and an observation chamber is filled with the liquid droplets. The observation chamber is preferably a plate for sedimentation.

(Step S204)

A fluorescent image of each of the liquid droplets with which the observation chamber is filled is acquired using a fluorescence microscope. The fluorescent image of the liquid droplet is acquired using an image pickup device such as a CCD camera mounted onto the fluorescence microscope. Through the acquisition of the fluorescent image of the liquid droplet, the fluorescence is detected.

(Step S205)

Based on the detection result of the fluorescence, the fluorescence intensity of each of the liquid droplets is determined, and each liquid droplet having a fluorescence intensity exceeding a predetermined threshold value is identified.

Each liquid droplet having a fluorescence intensity exceeding the predetermined threshold value may be identified based on a ratio between the fluorescence intensity of a reference liquid droplet and the fluorescence intensity of each of the liquid droplets containing the sample and the detection reagent. The “fluorescence intensity of a reference liquid droplet” means a fluorescence intensity acquired for a liquid droplet that is free of the target nucleic acid and contains the detection reagent.

The fluorescence intensity of each liquid droplet is determined by subjecting the fluorescent image of the liquid droplet taken with the image pickup device to predetermined image processing. For example, the fluorescence intensity of each liquid droplet may be determined by using Image J (manufactured by the American National Institutes of Health) or the like as image processing software.

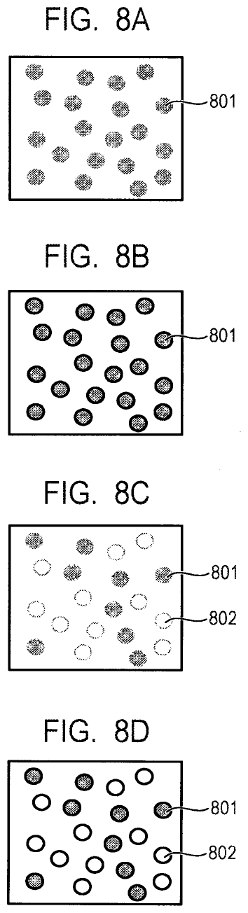

FIG. 8A is a schematic view for illustrating a fluorescent image (grayscale image) of reference liquid droplets, and FIG. 8B is a schematic view for illustrating an image obtained by binarizing the fluorescent image illustrated in FIG. 8A through the predetermined image processing to determine the fluorescence intensity. In addition, FIG. 8C is a schematic view for illustrating a fluorescent image (grayscale image) of liquid droplets each containing a sample and a detection reagent, and FIG. 8D is a schematic view for illustrating an image obtained by binarizing the fluorescent image illustrated in FIG. 8C through the predetermined image processing to determine the fluorescence intensity. The predetermined image processing has a function of binarizing a fluorescent image based on brightness information.

Negative liquid droplets 801 are liquid droplets free of the target nucleic acid, and are liquid droplets that do not generate fluorescence derived from the fluorescent substance possessed by the reporter molecule. In addition, positive liquid droplets 802 are liquid droplets containing the target nucleic acid, and are liquid droplets that generate fluorescence derived from the fluorescent substance possessed by the reporter molecule. The fluorescence intensity of each liquid droplet may be determined by binarizing the fluorescent image (grayscale image) through use of predetermined image processing.

(Step S206)

After the trans-cleavage reaction through the incubation in Step S202, the concentration of the target nucleic acid is calculated from the number of liquid droplets generating fluorescence. When the amount of the target nucleic acid contained in the sample is large, one liquid droplet may contain two or more molecules of the target nucleic acid. Thus, the number of molecules of the target nucleic acid may fail to match the number of liquid droplets generating fluorescence.

For the above-mentioned reason, it is preferred to calculate the concentration of the target nucleic acid through calculation that takes the Poisson distribution into consideration. In the Poisson distribution, when the average number of molecules per liquid droplet is set as λ, a ratio P(k) of the liquid droplets generating fluorescence can be expressed by the following equation (1).

P(k)=(λ^{k}/k!)e^{-λ}(k=0,1,2, . . . ) Equation (1)

From the number of liquid droplets generating fluorescence, P(k) can be determined and λ can be calculated. Thus, through use of the equation (1), the concentration of the target nucleic acid can be calculated from the number of liquid droplets in which fluorescence has been detected among all the liquid droplets.

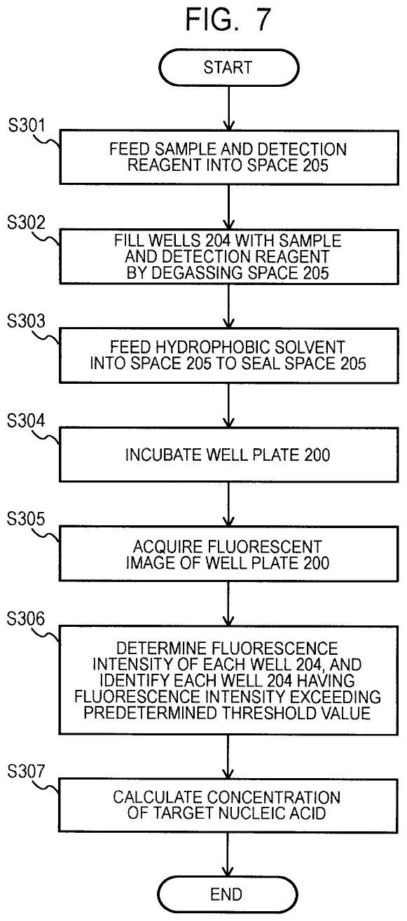

FIG. 7 is a flow chart for illustrating a flow of the method of detecting a nucleic acid according to the present invention in the case where the individual independent separated compartments are wells.

(Step S301)

As a well plate, the well plate 200 illustrated in FIG. 2A and FIG. 2B is used. In the well plate 200, the injection port portion (not shown) and the discharge port portion (not shown) are opened, and a reaction liquid formed of the sample and the detection reagent is fed from the injection port portion into the space 205.

(Step S302)

The wells 204 are filled with the reaction liquid. As a reaction liquid filling method, for example, there is given a method including leaving the well plate 200 under reduced pressure and degassing the space 205. Specifically, it is preferred to leave the well plate 200 in a vacuum desiccator at 0.1 atm for a predetermined time period. Through the degassing, the air in the wells 204 is removed, and thus the wells 204 can be efficiently filled with the reaction liquid. A degassing time period is not particularly limited, and may be freely set. The reaction liquid filling method is not limited to a method based on the degassing.

(Step S303)

The space 205 was fed with a hydrophobic solvent to be sealed. That is, the reaction liquid present in the space 205 above the wells 204 is replaced with the hydrophobic solvent. Examples of the hydrophobic solvent that may be used include a fluorine-based oil, a saturated aliphatic hydrocarbon, an unsaturated aliphatic hydrocarbon, an aromatic hydrocarbon, and a silicone oil. Examples of the fluorine-based oil may include Fluorinert (manufactured by 3M), AsahiKlin AE-3000 (manufactured by AGC Inc.), and Fomblin (manufactured by Solvay S.A.). As a saturated hydrocarbon, there may be given Isopar (manufactured by Exxon Mobil Corporation) or a mineral oil.

(Step S304)

The well plate 200 filled with the reaction liquid is incubated in an incubator at 37° C. However, the reaction temperature may be freely set, and is not limited to 37° C.

Through this incubation, a trans-cleavage reaction of CRISPR-Cas progresses, and fluorescence derived from a fluorescent substance possessed by the reporter molecule is generated.

(Step S305)

The incubation is ended after a reaction time set in advance, and a fluorescent image of each of the wells 204 is acquired using a fluorescence microscope.

(Step S306)

The fluorescence intensity of each of the wells 204 is determined, and each well 204 having a fluorescence intensity exceeding a predetermined threshold value is identified. Each well 204 having a fluorescence intensity exceeding the predetermined threshold value may be identified based on a ratio between the fluorescence intensity of a reference well and the fluorescence intensity of each of the wells 204 containing the sample and the detection reagent. The “fluorescence intensity of a reference well” means a fluorescence intensity acquired for a well 204 that is free of the target nucleic acid and contains the detection reagent.

Predetermined image processing is used for the determination of the fluorescence intensity of each of the wells 204. The above-mentioned Image J may be used as image processing software, and the fluorescence intensity of each of the wells 204 may be determined by an operation similar to that in the case where the individual independent separated compartments are liquid droplets.

(Step S307)

The concentration of the target nucleic acid may be calculated by an operation similar to that in the case where the individual independent separated compartments are liquid droplets.

(Other Step)

The method of detecting a nucleic acid in this embodiment may include a step other than the above-mentioned steps.

An example of the other step is a step of acquiring a fluorescent image serving as a reference (hereinafter abbreviated as “reference fluorescent image”).

For example, when fluorescence derived from a substance other than the fluorescent substance for detecting the nucleic acid is included in the fluorescent image acquired in Step S204 or Step S305, the concentration of the nucleic acid may not be correctly calculated. A conceivable example of the fluorescence of an origin other than the fluorescent substance for detecting the nucleic acid is fluorescence emitted by the wells.

In this case, the timing at which the reference fluorescent image is acquired in the step of acquiring a reference fluorescent image only needs to be before the presence of the nucleic acid causes the above-mentioned reporter molecule to be cleaved to emit fluorescence. For example, the reference fluorescent image may be acquired before the wells or the liquid droplets are filled with the sample or the detection reagent, before filling with a sealing oil for sealing the individual independent separated compartments, such as the wells or the liquid droplets, or before the incubation of Step S202 or Step S304 is performed (before heating is performed). In addition, when the timing at which the reference fluorescent image is acquired is after the wells or the liquid droplets have been filled with the detection reagent, and before incubation is performed (before heating is performed), the reference fluorescent image may be acquired after a predetermined time period from the time that the filling of the wells or the liquid droplets with the detection reagent is started. When the timing at which the reference fluorescent image is acquired is set to be before incubation is performed (before heating is performed), the reference fluorescent image may be acquired before a heating unit that has received a driving signal starts heating operation. In addition, the timing at which the reference fluorescent image is acquired may be determined by using a unit configured to monitor the state of the filling of the wells or the liquid droplets with the sample or the detection reagent.

The reference fluorescent image may be used as a reference in calculating the intensity of fluorescence based on the presence of the nucleic acid in each well or each liquid droplet in the fluorescent image obtained in Step S204 or Step S305. That is, when to what degree the fluorescence intensity is increased from the fluorescence intensity of the reference fluorescent image acquired in advance is calculated, the intensity of fluorescence derived from a component other than the fluorescent substance for detecting the nucleic acid in each well or each liquid droplet can be excluded. Thus, the fluorescence intensity derived from the fluorescent substance for detecting the nucleic acid can be more accurately obtained. In addition, for example, for the fluorescent image obtained in Step S204 or Step S305, it can be more correctly judged whether the pixel value (fluorescence intensity) of a well or liquid droplet of interest is equal to or more than the predetermined threshold value, or less than the predetermined threshold value. The predetermined threshold value only needs to be determined prior to the judgment of the number of the wells or the number of the liquid droplets. For example, the predetermined threshold value may be a value fixed in the nucleic acid detection apparatus, or may be a value to be set by input from a user. Being judged to be equal to or more than the predetermined threshold value may be expressed as being judged to be positive, and being judged to be less than the predetermined threshold value may be expressed as being judged to be negative.

In addition, for example, the reference fluorescent image may be acquired in order to detect a state in which a well or liquid droplet to be filled with the sample or the detection reagent has not been filled therewith owing to a failure or the like in the well or the liquid droplet, or in an operation of filling the well or the liquid droplet. In this case, for example, a well or a liquid droplet may be filled with a substance that emits light having a wavelength different from that of the fluorescent substance to be used for the detection of the nucleic acid (hereinafter referred to as “reference substance”), and a fluorescent image thereof may be acquired to acquire a fluorescence distribution. In this case, a difference between the center wavelength of the emission wavelength (fluorescence wavelength) of the reference substance and the center wavelength of the emission wavelength (fluorescence wavelength) of the fluorescent substance to be used for the detection of the nucleic acid is preferably 30 nm or more, more preferably 50 nm or more, still more preferably 100 nm or more.

When fluorescence is not detected as a result of the acquisition of the reference fluorescent image, it may be appropriate to start over from the first step, to display an error message, or to determine whether to start over or continue measurement in accordance with the number of wells or liquid droplets in which fluorescence is not detected. It may be appropriate not to count the fluorescence of the wells or the liquid droplets when the number of wells or liquid droplets in which fluorescence is not detected is equal to or less than a predetermined threshold value. The predetermined threshold value only needs to be determined prior to the judgment of the number of the wells or the number of the liquid droplets. For example, the predetermined threshold value may be a value fixed in the nucleic acid detection apparatus, or may be a value to be set by input from a user. In addition, for example, it may be appropriate to allow a user to appropriately make a choice regarding operation in the case where fluorescence is not detected as a result of the acquisition of the reference fluorescent image in the nucleic acid detection apparatus for carrying out the method of detecting a nucleic acid in this embodiment.

The nucleic acid detection apparatus for carrying out the method of detecting a nucleic acid in this embodiment may be configured to be switchable between a mode including the above-mentioned step of acquiring a reference fluorescent image and a mode not including the step of acquiring a reference fluorescent image.

As described above, the step of acquiring a reference fluorescent image has been described in the method of detecting a nucleic acid according to this embodiment, but may be applied to the nucleic acid detection apparatus according to the above-mentioned embodiment of the present invention. In this case, the reference fluorescent image may be acquired using the fluorescence detection unit or the image acquisition unit in the nucleic acid detection apparatus according to the embodiment of the present invention, or the reference fluorescent image may be acquired by another unit (e.g., a reference fluorescent image acquisition unit).

A program according to the present invention is a program for causing a computer included in a nucleic acid detection apparatus to execute the method of detecting a nucleic acid described above so as to cause the nucleic acid detection apparatus to execute the method.

### EXAMPLES

The present invention is described in more detail below by way of Examples. However, the present invention is by no means limited to Examples described below.

### Example 1

(Preparation of Reagents)

Preparation of Cas12a Stock Solution (400 nM)

As Cas12a, EnGen Lba Cas12a (Cpf1) (product name: M0653T, manufactured by NEB Inc., 100 μM) (hereinafter referred to simply as “Cas12a”) was used. The Cas12a was diluted with nuclease free water (product name: B1500S, manufactured by NEB Inc.) (hereinafter referred to simply as “purified water”) to prepare a Cas12a stock solution (400 nM).

Preparation of crRNA Stock Solution (500 nM)

As crRNA, Lb.Cas12a-crRNA1 (customized product, manufactured by Sigma-Aldrich, 100 μM) (hereinafter referred to simply as “crRNA”) was used. The crRNA was diluted with purified water to prepare a crRNA stock solution (500 nM).

The base sequence of crRNA is shown below (SEQ ID NO: 1).

uaauuucuacuaaguguagaugucuggccuuaauccaugcc

Preparation of DNA Solution

As a target nucleic acid, synthetic DNA (hereinafter referred to as “DNA_113 bp”) was used. DNA_113 bp was diluted with purified water to prepare a DNA stock solution (4 nM), and its concentration was checked through measurement with Qubit 2.0 Fluorometer (manufactured by Life Technologies).

The DNA stock solution (4 nM) was diluted with purified water to prepare a DNA solution (0.684 nM) (resulting in a final concentration of 0.171 nM). Further, ⅓ serial dilution of the DNA solution (0.684 nM) was performed with purified water to prepare a DNA solution 1 (0.228 nM), a DNA solution 2 (0.076 nM), and a DNA solution 3 (0.025 nM).

The base sequence of DNA_113 bp is shown below (SEQ ID NO: 2).

ctcacgccttatgactgcccttatgtcaccgcttatgtctcccgatatcacacccgttatctcagccctaatctctgcgg tttagtctggccttaatccatgcctcatagcta

Preparation of Reporter Molecule Solution (12 μM)

As a reporter molecule, a reporter molecule included in a commercially available kit (product name: DNaseAlert (trademark) Substrate Nuclease Detection System 11-02-01-04, manufactured by IDT) was used. This reporter molecule contains HEX serving as a fluorescent substance, and a quencher.

HiLyte (trademark) Fluor 488 (AnaSpec, Inc.) to be used as a standard fluorescent substance for recognizing liquid droplets to be described later was dissolved in purified water to prepare a standard fluorescent substance solution (800 nM). 50 pmol of the reporter molecule is contained per reporter molecule-containing tube included in the above-mentioned kit. Twelve of the reporter molecule-containing tubes were used, and the reporter molecule from the twelve tubes was dissolved in 50 μL of the standard fluorescent substance solution (800 nM). Thus, a reporter molecule solution (12 μM) was prepared.

(Preparation of Samples for forming Liquid Droplets)

Samples 1 to 4 for forming liquid droplets serving as individual independent separated compartments were prepared.

Specifically, first, 20 μL of the Cas12a stock solution (400 nM), 20 μL of the crRNA stock solution (500 nM), and 40 μL of any one of the DNA solutions 1 to 3 having respective concentrations prepared in the foregoing were mixed. Here, purified water was used in place of the DNA solution in the preparation of the sample 1. The resultant mixed solutions were each subjected to a reaction at 37° C. for 30 minutes to induce the formation of a Cas12a-crRNA-DNA complex.

Next, 20 μL of the reporter molecule solution (12 μM) and 20 μL of a 4×binding buffer having the following composition were mixed in a 1.5 mL microtube in advance.

Composition of 4× binding buffer: 80 mM Tris-HCl (pH 7.6), 400 mM KCl, 20 mM MgCl₂, 4 mM dithiothreitol (DTT), 20% glycerol, 200 μg/mL heparin

40 μL of the solution mixed and subjected to a reaction in the foregoing was added to the mixed solution to provide a sample for forming liquid droplets.

The concentrations of the DNA_113 bp contained in the samples 1 to 4 for forming liquid droplets prepared in the foregoing are shown below.
Sample 1: 0 pM
Sample 2: 6.3 pM
Sample 3: 19 pM
Sample 4: 57 pM

In addition, the respective concentrations of the components contained in the samples 1 to 4 for forming liquid droplets other than DNA are as follows: Cas12a: 50 nM, crRNA: 62.5 nM, reporter molecule: 3 μM, and HiLyte 488: 200 nM.

(Preparation of Liquid Droplets)

Liquid droplets were prepared by a pumping method with an SPG emulsification membrane using the samples 1 to 4 for forming liquid droplets prepared in the foregoing. A dispersed phase and a continuous phase are shown below.
Dispersed phase: each of the samples 1 to 4 for forming liquid droplets 80 μL
Continuous phase: An aliphatic hydrocarbon (product name: Isopar L, manufactured by Exxon Mobil Corporation) having a surfactant (product name: KF-6038, manufactured by Shin-Etsu Chemical Co., Ltd.) dissolved therein at a concentration of 4% 2.5 mL

An SPG pumping connector (pore diameter: 20 μm, manufactured by SPG Technology Co., Ltd.) was used as the SPG emulsification membrane, and the number of times of pumping was set to 10. Thus, liquid droplets each having a diameter of about 5 μm (a volume of about 65 fL) were obtained.

(Fluorescence Microscopic Observation)

The liquid droplets prepared in the foregoing were subjected to a reaction through incubation at 37° C. to promote the generation of fluorescence. After that, a plate for sedimentation (product name: MUR-300, manufactured by Matsunami Glass Ind., Ltd.) was filled therewith, followed by observation with a fluorescence microscope (product name: ECLIPS TE2000-U, manufactured by Nikon Corporation).

The observation of fluorescence derived from each fluorescent substance was performed under the following conditions to acquire a fluorescent image.
HEX: ex 533 nm, em 559 nm, EM gain 250
Standard fluorescent substance (HiLyte (trademark) Fluor 488): ex 499 nm, em 523 nm, EM gain 250

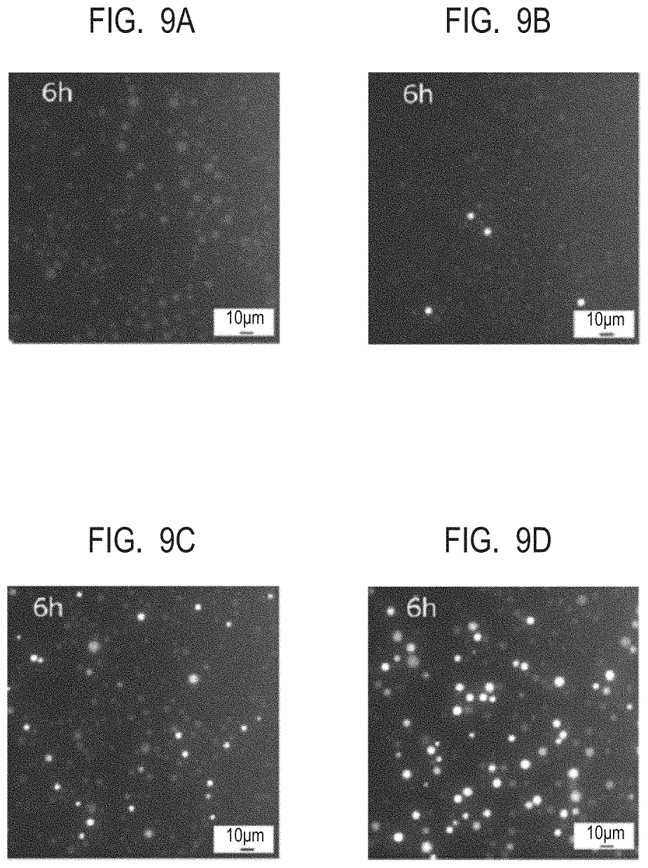

FIG. 9A to FIG. 9D show fluorescence microscope images acquired after the liquid droplets prepared using the respective samples were allowed to react for 6 hours. FIG. 9A, FIG. 9B, FIG. 9C, and FIG. 9D show fluorescence microscope images acquired for the sample 1, the sample 2, the sample 3, and the sample 4, respectively.

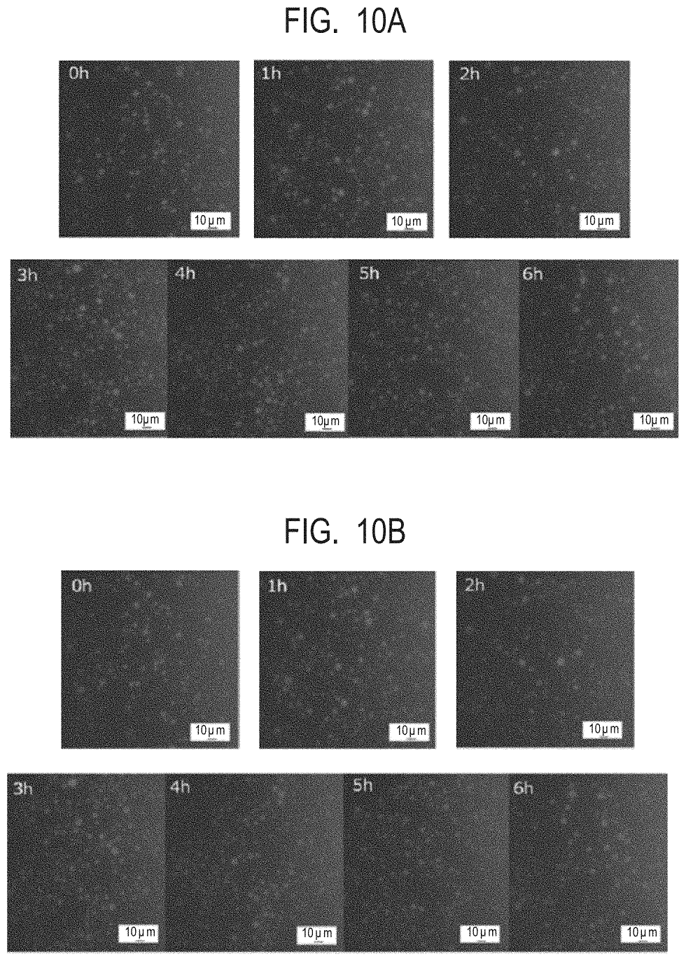

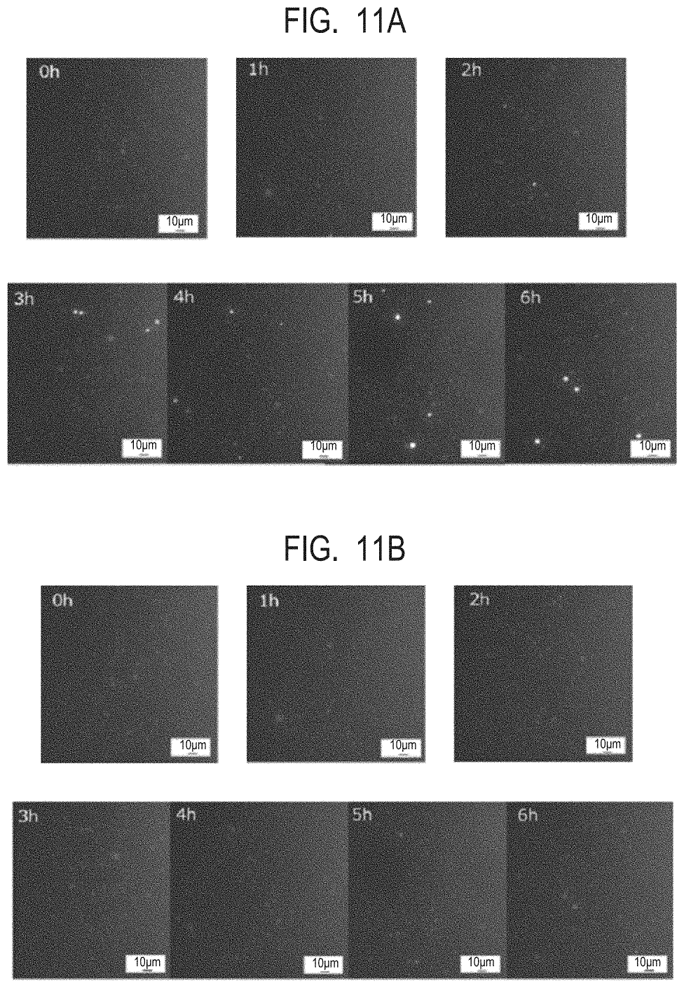

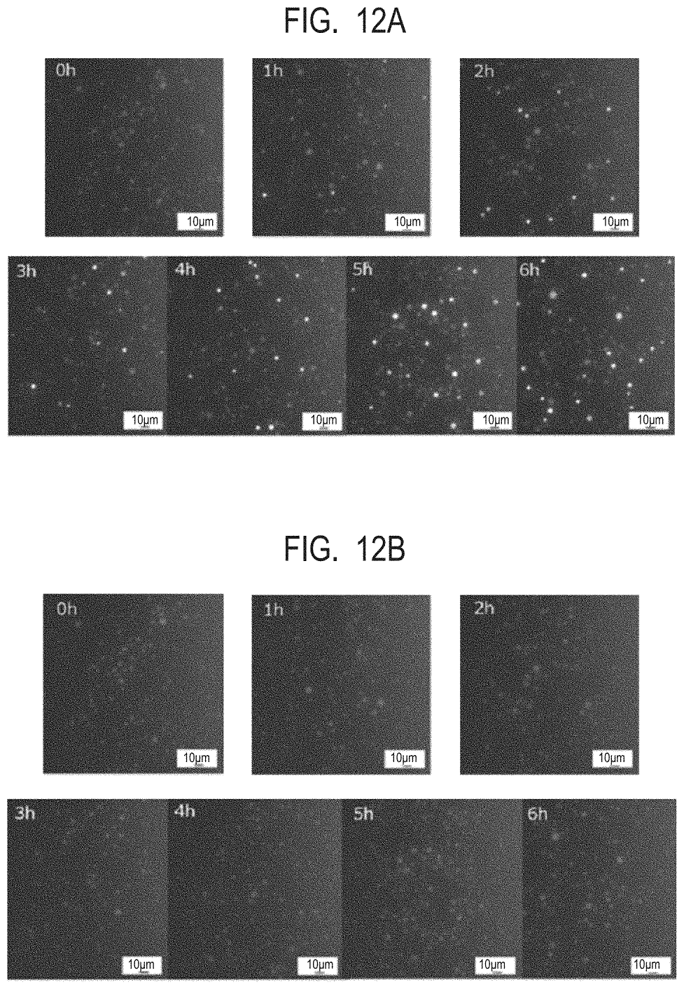

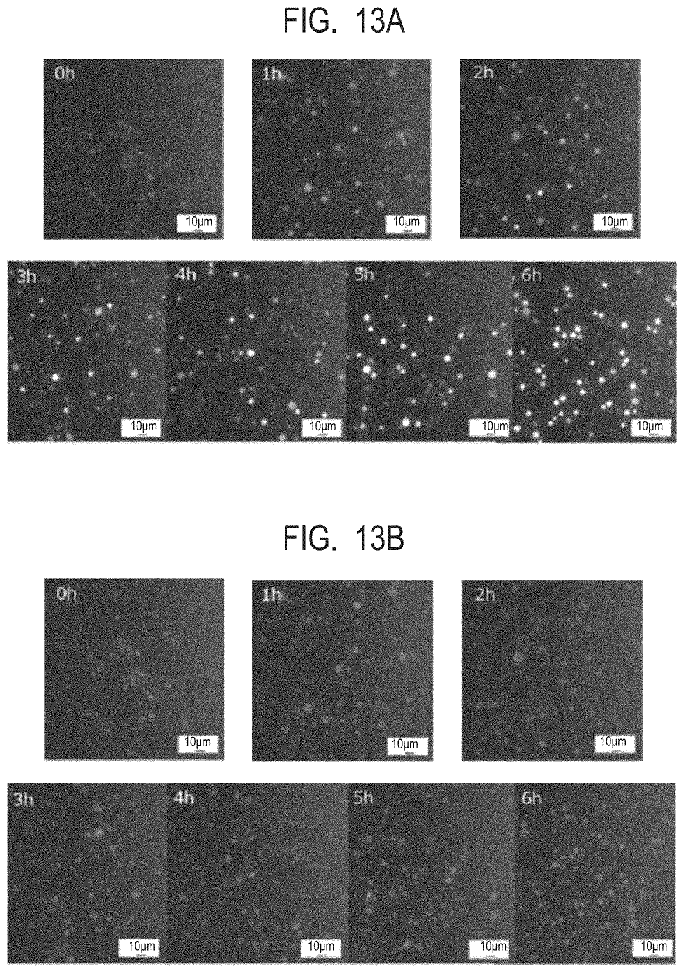

FIG. 10A to FIG. 13B are images showing temporal changes in fluorescence of the liquid droplets for the samples 1 to 4, respectively. FIG. 10A, FIG. 11A, FIG. 12A, and FIG. 13A each show a fluorescence microscope image containing both of the fluorescence of HEX derived from the reporter molecule and the background fluorescence of the reporter molecule. In addition, FIG. 10B, FIG. 11B, FIG. 12B, and FIG. 13B each show a fluorescence microscope image containing the fluorescence of the standard fluorescent substance.

As shown in FIG. 10B, FIG. 11B, FIG. 12B, and FIG. 13B, the liquid droplets were able to be recognized based on the fluorescence of the standard fluorescent substance. In addition, as shown in FIG. 11A, FIG. 11B, FIG. 12A, FIG. 12B, FIG. 13A, and FIG. 13B, it is found that, in the liquid droplets in which the fluorescence of HEX was observed, the fluorescence of the standard fluorescent substance is also observed. Thus, it was recognized that the liquid droplets were filled with the sample and the detection reagent, and the trans-cleavage reaction of Cas12a worked.

In addition, as shown in FIG. 9A to FIG. 13B, it was recognized that the number of positive liquid droplets increased in accordance with an increase in DNA concentration.

For example, as shown in FIG. 51 described in Japanese Patent Application Laid-Open No. 2020-501546, it was difficult in the related art to detect a target nucleic acid having a concentration as low as 6.3 pM by the CRISPR-Cas technology without performing an amplification step. The results of Example described above have shown that, in the present invention, a target nucleic acid having a concentration of 6.3 pM can be detected without undergoing the amplification step.

Next, through use of the fluorescence microscope images obtained in Example described above, the fluorescence intensity of each liquid droplet was determined, and each liquid droplet having a fluorescence intensity exceeding a predetermined threshold value was identified.

The liquid droplets obtained for the sample 1 were used as reference liquid droplets, and each liquid droplet having a fluorescence intensity exceeding the predetermined threshold value was identified based on a ratio between the fluorescence intensity of the reference liquid droplets and the fluorescence intensity of each liquid droplet containing the sample and the detection reagent. In this case, the fluorescence intensity of each liquid droplet containing the sample and the detection reagent is the fluorescence intensity of each liquid droplet obtained for the samples 2 to 4.

Specifically, the fluorescence intensity of each liquid droplet was determined using predetermined image processing (image processing software). The fluorescence intensity of each liquid droplet (negative liquid droplet) obtained for the sample 1 after 6 hours of reaction was 100. In addition, the fluorescence intensity of each positive liquid droplet among the respective liquid droplets obtained for the samples 2 to 4 after 6 hours of reaction fell within the range of from 200 to 250. The ratio was determined between the fluorescence intensity of each liquid droplet obtained for the samples 2 to 4 and the fluorescence intensity of each liquid droplet obtained for the sample 1 serving as the reference liquid droplet, and with the threshold value being set to 2, each liquid droplet in which the above-mentioned ratio exceeded 2 was identified as a positive liquid droplet.

Further, through use of the above-mentioned equation (1), the concentration of the target nucleic acid can be calculated from the total number of liquid droplets and the number of liquid droplets each having a fluorescence intensity exceeding the predetermined threshold value.

### Example 2

(Production of Wells)

The wells 204 illustrated in FIG. 2A and FIG. 2B were produced through a CYTOP application step, a photolithography step, and an etching/resist removal step.

In the CYTOP application step, a quartz substrate (synthetic quartz substrate, AQ grade, thickness: 1 mm, manufactured by AGC Inc.) was used as the lower substrate 201 and treated with a silane coupling agent (KBE-903, manufactured by Shin-Etsu Silicone), and then CYTOP (CTL-809A, manufactured by AGC Inc.) was applied.

In the photolithography step, a positive photoresist (AZ P4903, AZ Electronic Materials) was applied. Next, developing treatment with an alkali was performed by UV exposure from above through a photomask having a desired pattern. Only in portions that were UV-irradiated by the developing treatment, the photoresist dissolved to expose a hydrophobic resin layer.

In the etching/resist removal step, part of the resin layer was removed by etching with oxygen plasma through the photoresist part of which had been dissolved, to thereby form hydrophobic partition walls.

Finally, the photoresist was dissolved with an organic solvent to form the desired wells 204. The wells 204 had a diameter of 5 μm, a depth of 4 μm, and a pitch of 10 μm, and the number of wells was about 1,000,000.

(Production of Well Plate)

The well plate 200 illustrated in FIG. 2A and FIG. 2B was produced so as to include the lower substrate 201 having the above-mentioned wells 204 formed thereon, the upper substrate 202, an injection port portion (not shown), and a discharge port portion (not shown). Polycarbonate (thickness: 1 mm) was used as the upper substrate 202, and the upper substrate 202 was opposed to the openings of the wells 204 and the top surfaces of the partition walls 203 across the space 205. The distance in the space 205 from the top surface of each of the partition walls 203 to the upper substrate 202 was 250 μm.

(Preparation of Reaction Liquid for Wells)

A reaction liquid for filling the wells 204 was prepared. Specifically, first, 15 μL of the Cas12a stock solution (400 nM), 15 μL of the crRNA stock solution (500 nM), and 30 μL of the DNA solution 3 (0.025 μM) were mixed. The resultant mixed solution was subjected to a reaction at 37° C. for 30 minutes to induce the formation of a Cas12a-crRNA-DNA complex.

Next, 25 μL of the reporter molecule solution (12 μM) containing 800 nM HiLyte 488, 10 μL of Tween 20 (5%), 1 μL of BSA (30%), 4 μL of purified water, and 10 μL of a 10× binding buffer were mixed in a 1.5 mL microtube in advance. To the mixed solution, 50 μL of the solution mixed and subjected to a reaction in the foregoing was added to give a reaction liquid for wells. The final concentration of DNA was 6.3 pM.

(Filling of Wells with Reaction Liquid)

The reaction liquid was injected from the injection port portion on the upper substrate 202, and was fed so that the reaction liquid covered over the wells 204. Next, the well plate 200 was left to stand under reduced pressure to degas the space 205 to fill the wells with the reaction liquid. After that, the space 205 was fed with hydrophobic solvents to be sealed. Fluorine-based oils AsahiKlin AE-3000 (manufactured by AGC Inc.) and Fomblin Y-25 (manufactured by Solvay S.A.) were used as the hydrophobic solvents.

(Fluorescence Microscopic Observation)

The well plate prepared in the foregoing was subjected to a reaction through incubation at 37° C. to promote the generation of fluorescence. After that, observation was performed with a fluorescence microscope.

The observation of fluorescence derived from each fluorescent substance was performed under the following conditions to acquire a fluorescent image.
HEX: ex 533 nm, em 559 nm, EM gain 210
Standard fluorescent substance (HiLyte (trademark) Fluor 488): ex 499 nm, em 523 nm, EM gain 210

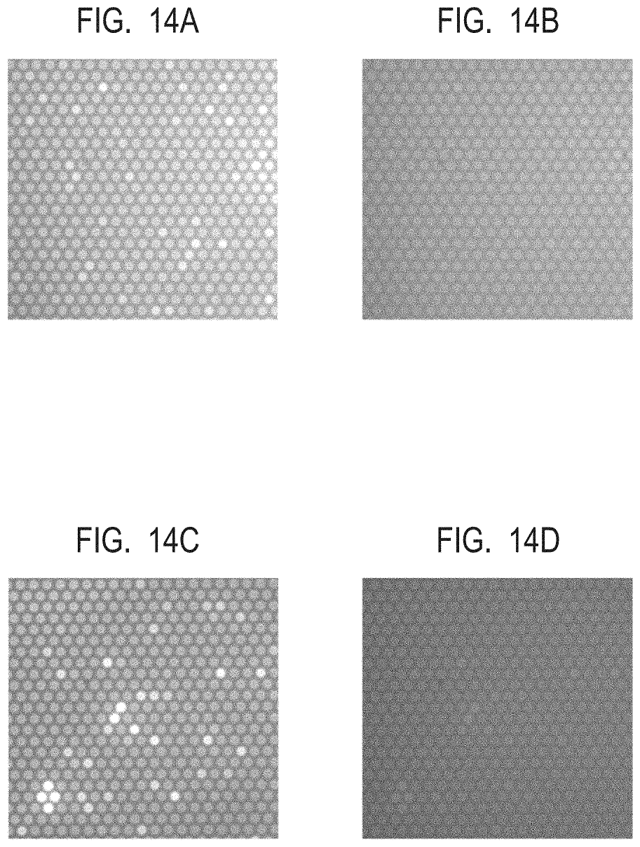

FIG. 14A to FIG. 14D show fluorescence microscope images of the wells. FIG. 14A shows a fluorescence microscope image containing both of the fluorescence of HEX derived from the reporter molecule and the background fluorescence of the reporter molecule after 2 hours of reaction. In addition, FIG. 14B shows a fluorescence microscope image containing the fluorescence of the standard fluorescent substance after 2 hours of reaction. In addition, FIG. 14C shows a fluorescence microscope image containing both of the fluorescence of HEX derived from the reporter molecule and the background fluorescence of the reporter molecule after 19 hours of reaction. In addition, FIG. 14D shows a fluorescence microscope image containing the fluorescence of the standard fluorescent substance after 19 hours of reaction.

As shown in FIG. 14B and FIG. 14D, the wells were able to be recognized based on the fluorescence of the standard fluorescent substance. In addition, as shown in FIG. 14A to FIG. 14D, it is found that, in the wells in which the fluorescence of HEX was observed, the fluorescence of the standard fluorescent substance is also observed. Thus, it was recognized that the wells were filled with the sample and the detection reagent, and the trans-cleavage reaction of Cas12a worked.

In addition, as shown in FIG. 51 described in Japanese Patent Application Laid-Open No. 2020-501546, it was difficult in the related art to detect a target nucleic acid having a concentration as low as 6.3 pM by the CRISPR-Cas technology without performing an amplification step. The results of Example described above have shown that, in the present invention, a target nucleic acid having a concentration of 6.3 pM can be detected without undergoing the amplification step.

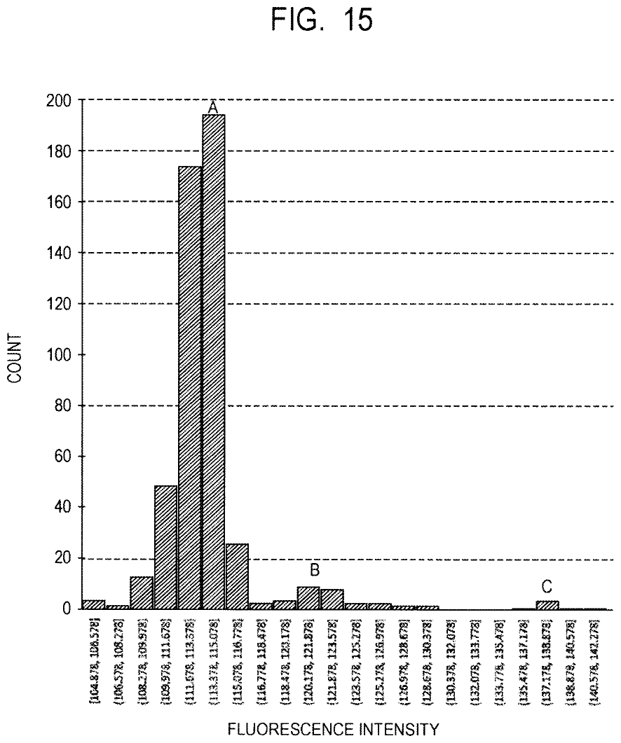

Next, the fluorescence microscope image after 19 hours of reaction of Example described above was subjected to predetermined image processing (image processing software) to give a histogram shown in FIG. 15.

The histogram shown in FIG. 15 shows the number of wells each showing a fluorescence intensity included in each fraction of fluorescence intensity obtained by dividing fluorescence intensities into intervals of a constant value.

In this histogram, as shown in FIG. 15, three peaks A, B, and C each corresponding to a category whose count number showed a local maximum value were observed. Of those, the peak A having the smallest fluorescence intensity is attributed to the background fluorescence of the reporter molecule.

In addition, wells falling within the category of the peak B were identified as positive wells B, and wells falling within the category of the peak C were identified as positive wells C. When the fluorescence intensities of the positive wells B and the positive wells C are compared, the fluorescence intensity of the positive wells C is stronger, and hence it is conceived that the positive wells B are filled with one DNA per well, and the positive wells C are filled with two DNAs per well.

Further, the concentration of the target nucleic acid can be calculated through use of the above-mentioned equation (1) from the total number of wells and the number of positive wells identified from the histogram.

### Example 3

(Preparation of Reaction Liquid for Wells)

A reaction liquid for filling the wells 204 was prepared. Specifically, first, 20 μL of the Cas12a stock solution (400 nM), 20 μL of the crRNA stock solution (500 nM), and 40 μL of the DNA solution 1 (0.228 μM) were mixed. The resultant mixed solution was subjected to a reaction at 37° C. for 30 minutes to induce the formation of a Cas12a-crRNA-DNA complex.

Next, 25 μL of the reporter molecule solution (12 μM) containing 800 nM HiLyte 488, 10 μL of Tween 20 (5%), 1 μL of BSA (30%), 4 μL of an aqueous spermine solution (50 mM), and 10 μL of a 10× binding buffer were mixed in a 1.5 mL microtube in advance. To the mixed solution, 50 μL of the solution mixed and subjected to a reaction in the foregoing was added to give a reaction liquid for wells. The final concentration of DNA was 57 pM.

(Filling of Wells with Reaction Liquid)

Filling of wells with the reaction liquid was performed by the same operation method as in Example 2.

(Fluorescence Microscopic Observation)

The well plate prepared in the foregoing was subjected to a reaction through incubation at 37° C. to promote the generation of fluorescence. After that, observation was performed with a fluorescence microscope.

The observation of fluorescence derived from each fluorescent substance was performed under the following conditions to acquire a fluorescent image.
HEX: ex 533 nm, em 559 nm, EM gain 210
Standard fluorescent substance (HiLyte (trademark) Fluor 488): ex 499 nm, em 523 nm, EM gain 210

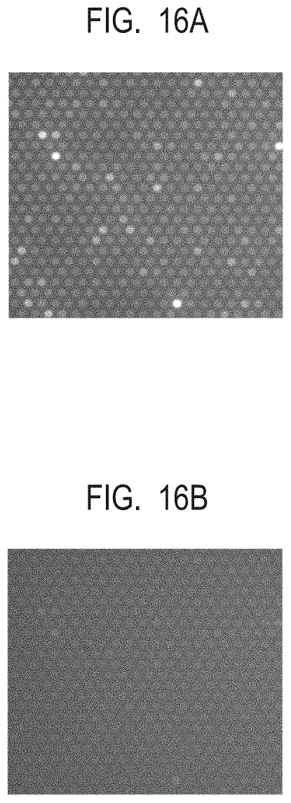

FIG. 16A and FIG. 16B show fluorescence microscope images acquired after 0.5 hour of reaction. FIG. 16A shows a fluorescence microscope image containing both of the fluorescence of HEX derived from the reporter molecule and the background fluorescence of the reporter molecule. In addition, FIG. 16B shows a fluorescence microscope image containing the fluorescence of the standard fluorescent substance.

As shown in FIG. 16B, the wells were able to be recognized based on the fluorescence of the standard fluorescent substance. In addition, as shown in FIG. 16A and FIG. 16B, it is found that, in the wells in which the fluorescence of HEX was observed, the fluorescence of the standard fluorescent substance is also observed. Thus, it was recognized that the wells were filled with the sample and the detection reagent, and the trans-cleavage reaction of Cas12a worked.

In addition, in Example 3, in which a reaction liquid containing spermine was used, the fluorescence of HEX was able to be observed in 0.5 hour. Meanwhile, in Example 2, the fluorescence of HEX was not able to be recognized after 0.5 hour. Thus, it was recognized that, when the detection reagent contained spermine, the trans-cleavage reaction of Cas12a was able to be promoted to shorten the detection time as compared to Example 2, in which a detection reagent containing no amino compound was used.

### Example 4

(Production of Composite Particles in which Complex of Cas12a and crRNA is Bound to Particles)

First, a magnetic particle (Magnosphere (trademark) MS300/Carboxyl) dispersion was placed in a microtube, and magnetic particles were settled with a magnet. After the supernatant had been removed, magnetic particle pellets were dispersed again by adding IVIES buffer solution (100 mM, pH 5.4) thereto, and N-hydroxysulfosuccinimide (sulfo-NHS) and water-soluble carbodiimide (WSC) were added. After that, the mixture was stirred at 25° C. for 1 hour, and the magnetic particles were recovered with a magnet.

Subsequently, the recovered magnetic particles were washed with IVIES buffer solution and dispersed with MES buffer solution, and an arbitrary amount of an anti-His tag antibody (Anti-His-tag mAb, MBL Life Science) was added. After that, the mixture was stirred at 25° C. for 2 hours.

Subsequently, a large excess of ethanolamine was added to inactivate active groups on the surfaces of the magnetic particles. The magnetic particles were recovered with a magnet, and the recovered magnetic particles were washed with IVIES buffer solution to produce antibody-immobilized particles.

A storage buffer (10 mM HEPES-NaOH (pH 7.9), 50 mM KCl, 1 mM EDTA, 10% glycerol) was added to the resultant antibody-immobilized particles to prepare an antibody-immobilized particle liquid. The antibody-immobilized particle liquid was stored at 4° C. until use.

Next, the diluted Cas12a and crRNA were mixed at a concentration ratio (molar ratio) of 1:1.25, and the mixture was incubated at 37° C. for 30 minutes to produce a Cas12a-crRNA complex.

In addition, the produced antibody-immobilized particle liquid (1 wt %) was aliquoted into a 2 mL sample tube (manufactured by VIOLAMO, model number: 1-1600-04). After stirring, the sample tube was allowed to stand on a magnetic stand (Magical Trapper, manufactured by TOYOBO, model number: MGS-101) and left at rest for 1 minute, and then the supernatant was removed to remove the solution. 0.05% Tween 20-containing PBS (PBS-T) was added as a particle washing liquid, and after stirring, the solution was removed in the same manner as in the foregoing. The above-mentioned operation was repeated twice, followed by washing.

The antibody-immobilized particles after the washing were suspended in PBS-T, and the Cas12a-crRNA solution prepared in the foregoing was added so as to have an arbitrary concentration. After stirring, the mixture was subjected to a reaction on a shaker for 1 hour. In this case, the Cas12a used has a His tag at the N-terminus, and hence the Cas12a is bound to the antibody-immobilized particles through an antigen-antibody reaction between the His tag possessed by the Cas12 and the anti-His tag antibody possessed by each of the antibody-immobilized particles. Thus, composite particles in which the complex of Cas12a and crRNA was bound to the particles were produced.

After the reaction, the solution was removed, and a washing operation was performed with PBS-T. After the washing, the resultant was suspended in purified water, and the suspension was stirred and then stored at 4° C. until use.

(Preparation of Reaction Liquid for Wells)

A reaction liquid for filling the wells 204 was prepared. Specifically, first, 7.3 μL of the composite particles (3.1×10⁸ particles/mL) produced in the foregoing, 22.7 μL of water, and 30 μL of the DNA solution 1 (0.228 μM) were mixed. The resultant mixed solution was subjected to a reaction at 37° C. for 30 minutes to form a complex of the composite particles and DNA.

Next, the following materials were prepared.
Reporter molecule solution (12 μM) containing 800 nM HiLyte 488: 25 μL
Tween 20 (5%): 10 μL
BSA (30%): 1 μL
Aqueous spermine solution (50 mM): 4 μL
10× Binding buffer: 10 μL

Those materials were mixed in a 1.5 mL microtube in advance. To the mixed solution, 50 μL of the above-mentioned complex of the composite particles and DNA was added to give a reaction liquid for wells. The final concentration of DNA was 57 pM.

(Filling of Wells with Reaction Liquid)

Filling of wells with the reaction liquid was performed by the same operation method as in Example 2.

(Fluorescence Microscopic Observation)

The well plate prepared in the foregoing was subjected to a reaction through incubation at 37° C. to promote the generation of fluorescence. After that, observation was performed with a fluorescence microscope.

The observation of fluorescence derived from each fluorescent substance was performed under the following conditions to acquire a fluorescent image.
HEX: ex 533 nm, em 559 nm, EM gain 210
Standard fluorescent substance (HiLyte (trademark) Fluor 488): ex 499 nm, em 523 nm, EM gain 210

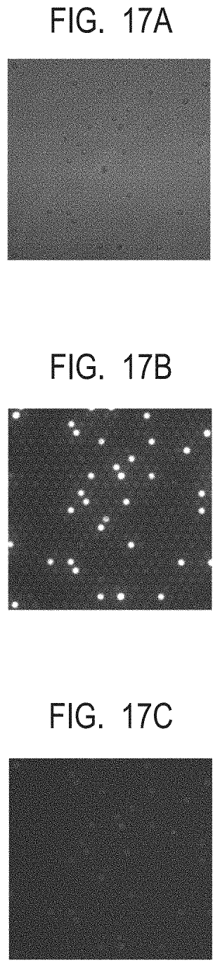

FIG. 17A to FIG. 17C show a bright-field image and fluorescence microscope images of the wells acquired after 1 hour of reaction. FIG. 17A shows a bright-field image of the wells. FIG. 17B shows a fluorescence microscope image containing both of the fluorescence of HEX derived from the reporter molecule and the background fluorescence of the reporter molecule. In addition, FIG. 17C shows a fluorescence microscope image containing the fluorescence of the standard fluorescent substance.

It was able to be recognized from FIG. 17A that the wells were filled with the particles.

In addition, it is found from FIG. 17A to FIG. 17C that the fluorescence of HEX is observed from the wells filled with the particles. Thus, it was recognized that the DNA was captured by Cas12a bound to the particles, and the trans-cleavage reaction of Cas12a worked. The DNA concentration in this Example, i.e., 57 pM is a concentration at which all the individual independent separated compartments are predicted to be positive.

### Example 5

(Immobilization of Anti-His Tag Antibody onto Magnetic Particles) A magnetic particle (Magnosphere (trademark) MS300/Carboxyl) dispersion was placed in a microtube, and magnetic particles were settled with a magnet. After the supernatant had been removed, magnetic particle pellets were dispersed again by adding IVIES buffer solution (100 mM, pH 5.4) thereto, and N-hydroxysulfosuccinimide (sulfo-NHS) and water-soluble carbodiimide (WSC) were added. The mixture was stirred at 25° C. for 1 hour, and the magnetic particles were recovered with a magnet.

The recovered magnetic particles were washed with IVIES buffer solution and dispersed with IVIES buffer solution, and an arbitrary amount of an anti-His tag antibody (Anti-His-tag mAb, MBL Life Science) was added. The mixture was stirred at 25° C. for 2 hours. Subsequently, two cases, i.e., cases of performing and not performing a blocking operation were arranged. In the case of performing the blocking operation, a PEG amine having a molecular weight of 5,000 was added in a large excess with respect to carboxy groups on the surfaces of the magnetic particles, and the mixture was stirred at room temperature for 45 minutes.

No matter whether with or without blocking, a large excess of ethanolamine was then added to inactivate active groups on the surfaces of the particles. The magnetic particles were recovered with a magnet, and the recovered magnetic particles were washed with IVIES buffer solution to produce antibody-immobilized particles (with and without blocking).

A storage buffer (10 mM HEPES-NaOH (pH 7.9), 50 mM KCl, 1 mM EDTA, 10% glycerol) was added to the antibody-immobilized particles to prepare an antibody-immobilized particle liquid, which was stored at 4° C. until use.

(Reaction between Cas12a-crRNA and Antibody-immobilized Particles)

The diluted Cas12a and crRNA were mixed at a concentration ratio (molar ratio) of 1:1.25, and the mixture was incubated at 37° C. for 30 minutes to produce a Cas12a-crRNA complex.

The produced antibody-immobilized particle liquid (1 wt %) was aliquoted into a 2 mL sample tube (manufactured by VIOLAMO, model number: 1-1600-04). After stirring, the sample tube was allowed to stand on a magnetic stand (Magical Trapper, manufactured by TOYOBO, model number: MGS-101) and left at rest for 1 minute, and then the supernatant was removed to remove the solution. PBS-T was added as a particle washing liquid, and after stirring, the solution was removed in the same manner as in the foregoing. The above-mentioned operation was repeated twice.

The particles were suspended in PBS-T, and the Cas12a-crRNA solution prepared in the foregoing was added so as to have an arbitrary concentration. After stirring, the mixture was subjected to a reaction on a shaker for 1 hour. After the reaction, the solution was removed, and a washing operation was performed with PBS-T. After the washing, the resultant was suspended in purified water to produce composite particles in which the Cas12a-crRNA complex was bound to the antibody-immobilized particles. After stirring, the suspension was stored at 4° C. until use. Of the resultant composite particles, the particles that underwent the blocking operation are referred to as “composite particles AntBL”, and the particles that did not undergo the blocking operation are referred to as “composite particles Ant”.

(Reaction between Cas12a-crRNA and Nickel Particles)

The diluted Cas12a and crRNA were mixed at a concentration ratio (molar ratio) of 1:1.25, and the mixture was incubated at 37° C. for 30 minutes to produce a Cas12a-crRNA complex.

A commercially available nickel particle liquid (PureProteome (trademark) Nickel Magnetic Beads, Merck, 3 wt %) was aliquoted into a 2 mL sample tube (manufactured by VIOLAMO, model number: 1-1600-04). After stirring, the sample tube was allowed to stand on a magnetic stand (Magical Trapper, manufactured by TOYOBO, model number: MGS-101) and left at rest, and the supernatant was then removed to remove the solution.

PBS-T was added as a particle washing liquid, and after stirring, the solution was removed. The above-mentioned operation was repeated twice. The particles were suspended in PBS-T, and the Cas12a solution prepared in the foregoing was added so as to have an arbitrary concentration. After stirring, the mixture was subjected to a reaction on a shaker for 1 hour. After the reaction, the solution was removed, and a washing operation was performed with PBS-T. After the washing, the resultant was suspended in purified water to produce composite particles in which the Cas12a-crRNA complex was immobilized onto nickel particles. After stirring, the suspension was stored at 4° C. until use. The resultant composite particles were named “composite particles Ni”.

(Evaluation of Activity of Composite Particles in Wells)

The activity of the composite particles produced in the foregoing under conditions with and without the addition of an amino compound was evaluated using a 96-well plate (Thermo Fisher Scientific, model number: 137101).

Each type of particles and the DNA solution were mixed in water so that the final concentration of Cas12a was 10 nM, and that the final concentration of DNA was 2 nM, and the mixture was left at rest at 37° C. for 30 minutes or more to produce a complex with DNA.

After that, the following materials were prepared.
Complex of each type of particles and DNA (in such an amount that the final concentration of Cas12a becomes 5 nM in the cases of the composite particles AntBL and the composite particles Ant, and such an amount that the final concentration of Cas12a becomes 3.7 nM in the case of the composite particles Ni)
125 nM Reporter molecule
8 nM HiLyte 488 serving as an internal standard dye
2 mM Spermine (Nacalai Tesque, Inc., model number: 32111-31) serving as an amino compound

Those materials were added to a reaction buffer in the 96-well plate, and the total volume was adjusted to 80 μL. In the condition without the addition of the amino compound, spermine in the above-mentioned materials was not used. In addition, as the reaction buffer, two cases, i.e., a case of using a binding buffer and a case of using NEBuffer (trademark) were performed.

After that, a fluorescence intensity was measured with a fluorescence plate reader (Synergy MX, manufactured by BioTek Instruments, Inc.) at 37° C. every 2 min for 2 hours. For measurement wavelengths, an excitation wavelength of 485±20 nm and a fluorescence wavelength of 528±20 nm were used for HiLyte 488. An excitation wavelength of 535±20 nm and a fluorescence wavelength of 595±20 nm were used for the reporter molecule.

For the binding buffer, a 10× binding buffer was prepared, and was used by being added in an amount one tenth as large as that of the reaction solution.

For NEBuffer (trademark) 2.1, 10×NEBuffer (trademark) 2.1 included with EnGen Lba Cas12a (Cpf1) (NEB Inc., M0653T) was used by being added in an amount one tenth as large as that of the reaction solution.

The resultant fluorescence intensity was evaluated as the ratio of the fluorescence intensity of the reporter molecule to the fluorescence intensity of the internal standard dye (fluorescence intensity of reporter molecule/fluorescence intensity of internal standard dye).

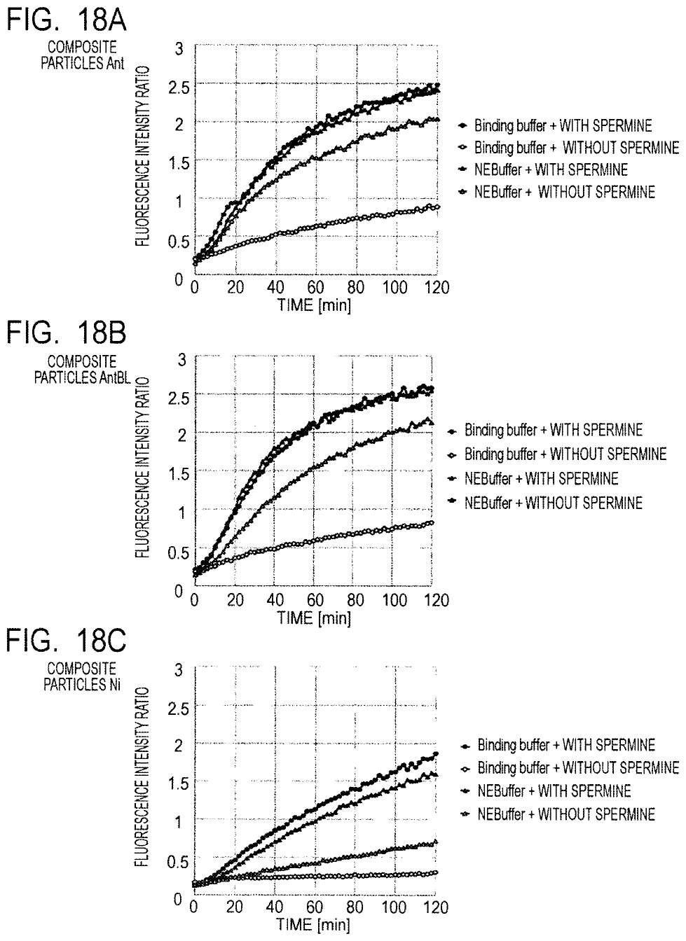

FIG. 18A to FIG. 18C show temporal changes in fluorescence intensity ratios (fluorescence intensity of reporter molecule/fluorescence intensity of internal standard dye) in the cases of using the respective composite particles. FIG. 18A shows the results in the case of using the composite particles Ant, FIG. 18B shows the results in the case of using the composite particles AntBL, and FIG. 18C shows the results in the case of using the composite particles Ni.

As shown in FIG. 18A to FIG. 18C, it is found that, whichever composite particles or whichever reaction buffer was used, the reaction was promoted by adding spermine as an amino compound.

### Example 6

(Preparation of 500 nM crRNA)

crRNA (referred to as “crRNA_T790M”) included in EnGen Lba Cas12a (Cpf1) (NEW ENGLAND BioLabs (NEB Inc.), M0653T) was diluted with purified water to prepare a 500 nM storage solution.

The sequence of crRNA_T790M is shown below (SEQ ID NO: 3).

uaauuucuacuaaguguagauaucaugcagcucaugcc

(Production of Composite Particles Having Cas12a-crRNA Complex Immobilized Thereonto)

The diluted Cas12a and crRNA were mixed at a concentration ratio (molar ratio) of 1:1.25, and the mixture was incubated at 37° C. for 30 minutes to produce a Cas12a-crRNA complex.

Anti-His tag antibody particles (Anti-His-tag mAb-Magnetic Beads (trademark), MBL Life Science, 1 wt %) were aliquoted into a 2 mL sample tube (manufactured by VIOLAMO, model number: 1-1600-04). After stirring, the sample tube was allowed to stand on a magnetic stand (Magical Trapper, manufactured by TOYOBO, model number: MGS-101) and left at rest for 1 minute, and the supernatant was removed to remove the solution. 0.5% Tween 20-containing PBS was added as a particle washing liquid, and after stirring, the solution was removed. The above-mentioned operation was repeated twice.

The particles were suspended in 0.5% Tween 20-containing PBS, and the Cas12a-crRNA complex prepared in the foregoing was added so as to have an arbitrary concentration. After stirring, the mixture was subjected to a reaction on a shaker for 1 hour. After the reaction, the solution was removed, and a washing operation was performed with purified water. After the washing, the resultant was suspended in purified water to produce composite particles. After stirring, the suspension was stored at 4° C. until use.

(Preparation of DNA)

100% EGFR wildtype (50 ng/μL, Riken Genesis Co., Ltd., model number: HD709) and 50% EGFR T790M (50 ng/μL, Riken Genesis Co., Ltd., model number: HD258), both being genomic DNA, were each used as a template in PCR.

A product amplified by PCR using 100% EGFR wildtype as the template was used as contaminating DNA_WT. In addition, a product amplified by PCR using 50% EGFR T790M as the template was used as target DNA_T790M. Each PCR product was measured for its concentration with Qubit 2.0 Fluorometer (manufactured by Life Technologies) after purification.

Subsequent concentration calculation for the product amplified by PCR using 50% EGFR T790M as the template was performed on the assumption of having the same mutant allele ratio as 50% EGFR T790M.

The PCR products were each diluted with purified water to prepare a 4 nM storage solution. Further, the 4 nM storage solution was diluted with purified water to prepare DNA solutions having various concentrations.

The sequences of primers used in the PCR are shown below.

EGFR primer (Forward)
(SEQ ID NO: 4)
tcacctccaccgtgcatttcatca
EGFR primer (Reverse)
(SEQ ID NO: 5)
ttgcgatctgcacacaccagttga

In addition, the sequences of contaminating DNA_WT and target DNA_T790M as the PCR products are shown below. The underlined part represents the target sequence of the crRNA.

DNA_WT
(SEQ ID NO: 6)
tcacctccaccgtgcatttcatcacgcagctcatgcccttcggctgcctc
ctggactatgtccgggaacacaaagacaatattggctcccagtacctgct
caactggtgtgtgcagatcgcaa
DNA_T790M
(SEQ ID NO: 7)
tcacctccaccgtgcatttcatcatgcagctcatgcccttcggctgcctc
ctggactatgtccgggaacacaaagacaatattggctcccagtacctgct
caactggtgtgtgcagatcgcaa

Subsequently, the composite particles produced in the foregoing were evaluated for efficiency with which DNA was recovered and concentrated.

First, the following materials were prepared.
Composite particles each having a number of Cas12a immobilized of 6.7×10⁵ per particle (referred to as “composite particles A”): 2.7×10⁻¹¹ mol
Composite particles each having a number of Cas12a immobilized of 1.5×10⁴ per particle (referred to as “composite particles B”): 6.0×10⁻¹³ mol
Target DNA_T790M: 1.5×10⁻¹³ mol
DNA_WT serving as contaminating DNA: 1.5×10⁻¹² mol
Tween 20: final concentration: 0.5%

The composite particles A or B, target DNA_T790M, DNA_WT, and Tween 20 were added to a 2 mL tube (Eppendorf), and the total volume of the solution was adjusted to 1.1 mL. The resultant mixed solution was shaken at room temperature for 1 hour. After that, the particles were recovered with a magnet, and the supernatant was removed, followed by washing with 1 mL of purified water. After that, the particles were recovered with a magnet again, and the supernatant was removed. The residue was suspended in 50 μL of purified water to provide a DNA-recovered particle solution.

In addition, as a case of not containing the contaminating DNA in the foregoing, the same operations were performed without the use of DNA_WT.

Subsequently, the following materials were prepared.
40 μL of DNA-recovered particle solution (final concentration of composite particles: 4×10⁻¹³ M)
Reporter molecule (final concentration: 125 nM)
8 nM HiLyte 488 serving as an internal standard dye
2 mM Spermine

Those materials were added to the binding buffer in a 96-well plate (Thermo Fisher Scientific, model number: 137101), and the total volume was adjusted to 80 μL. After that, a fluorescence intensity was measured with a fluorescence plate reader Synergy MX (BioTek Instruments, Inc.) at 37° C. every 2 min for 2 hours.

For measurement wavelengths, an excitation wavelength of 485±20 nm and a fluorescence wavelength of 528±20 nm were used for HiLyte 488. In addition, an excitation wavelength of 535±20 nm and a fluorescence wavelength of 595±20 nm were used for the reporter molecule.

For comparison in evaluating DNA recovery efficiency, target DNA_T790M was added without being subjected to the recovery operation, and a fluorescence intensity was measured in the same manner as in the foregoing.

Specifically, the composite particles A or B were set to have the same molar concentration as in the foregoing (final concentration of composite particles: 4×10⁻¹³ M), and target DNA_T790M was set to have a final concentration of 1.5 nM, assumed in the case of achieving 100% recovery. In addition to those materials, the reporter molecule (final concentration: 125 nM), 8 nM HiLyte 488, and 2 mM spermine were added to the binding buffer in the 96-well plate, and the total volume was adjusted to 80 μL. After that, a fluorescence intensity was measured in the same manner as in the foregoing.

The resultant fluorescence intensity was evaluated as the ratio of the fluorescence intensity of the reporter molecule to the fluorescence intensity of the internal standard dye (fluorescence intensity of reporter molecule/fluorescence intensity of internal standard dye).

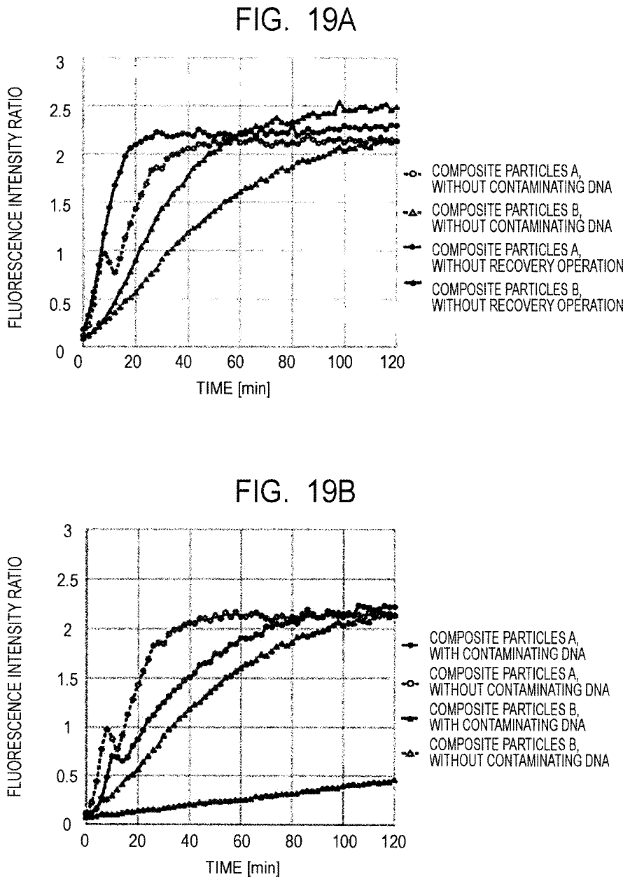

FIG. 19A shows temporal changes in fluorescence intensity ratios in the cases of performing and not performing the recovery operation under the condition without the contaminating DNA. In addition, FIG. 19B shows temporal changes in fluorescence intensity ratios in the case of performing the recovery operation under each of the conditions with and without the contaminating DNA.

As shown in FIG. 19A, it was able to be recognized that, whichever composite particles were used, DNA had been able to be recovered and concentrated with a high recovery ratio. In addition, it was suggested that, as the number of composite particles immobilized increased, the recovery ratio tended to be higher.

In addition, as shown in FIG. 19B, it was able to be recognized that, even when the contaminating DNA coexisted, the composite particles were capable of recovering and concentrating DNA, and that as the number of composite particles immobilized increased, the recovery ratio of DNA became higher.

Examples described above have shown that, according to the present invention, when the detection reagent contains an amino compound, the trans-cleavage reaction of the effector protein can be promoted to shorten the detection time. In addition, it has been shown that, for example, even a target nucleic acid to be discarded without being caused to fill individual independent separated compartments can be recovered by allowing the composite particles, in which the complex of the effector protein and crRNA is bound to particles, to act on the target nucleic acid. The recovered target nucleic acid can be detected by filling the individual independent separated compartments with the composite particles that have captured the target nucleic acid.

The embodiments of the present invention may be realized by a method to be executed by a computer of a system or an apparatus, by executing one or a plurality of functions of the above-mentioned embodiments with a computer of a system or apparatus (e.g., an application-specific integrated circuit (ASIC)) configured to read and execute a computer-executable instruction (e.g., one or more programs) recorded on a storage medium, and/or by reading and executing a computer-executable instruction from a storage medium with a computer of a system or apparatus including one or a plurality of circuits configured to execute one or a plurality of functions of the above-mentioned embodiments and for the purpose of, for example, executing one or a plurality of functions of the above-mentioned embodiments, and/or by controlling one or a plurality of circuits to execute one or a plurality of functions of the above-mentioned embodiments. The computer may include one or more processors (e.g., a central processing unit (CPU) or a microprocessor unit (MPU)), and may include a network of individual computers or individual processors to read and execute the computer-executable instruction. The computer-executable instruction may be, for example, provided from the network or the storage medium to the computer. The storage medium may include one or more of, for example, a hard disk, a random access memory (RAM), a read only memory (ROM), a storage apparatus of a distributed computing system, an optical disc (e.g., a compact disc (CD), a digital versatile disc (DVD), or a Blu-ray Disc (BD)), a flash memory device, and a memory card.

According to the present invention, the nucleic acid detection apparatus and method of detecting a nucleic acid, for detecting a target nucleic acid having a low concentration in a simple manner, can be provided.

The present invention is not limited to the embodiments described above, and various changes and modifications may be made without departing from the spirit and scope of the present invention. The following claims are appended hereto in order to make the scope of the present invention public.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A nucleic acid detection apparatus comprising:
a distribution unit configured to distribute a sample and a detection reagent to a plurality of individual independent separated compartments, the sample containing a target nucleic acid, and the detection reagent containing an effector protein, crRNA to be bound to the target nucleic acid, and a reporter molecule;
an activation unit configured to activate the effector protein through binding of the crRNA to the target nucleic acid;
a fluorescence generation unit configured to modify the reporter molecule with the activated effector protein to generate fluorescence;
a fluorescence detection unit configured to detect the fluorescence; and
an identification unit configured to determine, based on a detection result obtained with the fluorescence detection unit, a fluorescence intensity of each of the individual independent separated compartments, and to identify each of the individual independent separated compartments having a fluorescence intensity exceeding a predetermined threshold value.

2. The nucleic acid detection apparatus according to claim 1, wherein the fluorescence detection unit is an image acquisition unit configured to acquire an image containing the individual independent separated compartments.

3. The nucleic acid detection apparatus according to claim 2, wherein the identification unit is configured to identify each of the individual independent separated compartments having a fluorescence intensity exceeding the predetermined threshold value by processing the image acquired with the image acquisition unit.

4. The nucleic acid detection apparatus according to claim 1, wherein the effector protein is any one of Cas12 or Cas13.

5. The nucleic acid detection apparatus according to claim 1, wherein the detection reagent further contains an amino compound.

6. The nucleic acid detection apparatus according to claim 5, wherein the amino compound has —NH₂.

7. The nucleic acid detection apparatus according to claim 6, wherein the amino compound has one or more —NH₂'s and one or more —NH—'s.

8. The nucleic acid detection apparatus according to claim 7, wherein the amino compound is spermine.

9. The nucleic acid detection apparatus according to claim 1, wherein the effector protein is bound to a particle.

10. The nucleic acid detection apparatus according to claim 9, wherein a binding portion between the effector protein and the particle has a structure derived from a carboxy group bonded to the particle.

11. The nucleic acid detection apparatus according to claim 9, wherein the effector protein is bound via an N-terminus thereof to the particle.

12. The nucleic acid detection apparatus according to claim 9, wherein the effector protein and the particle are bound to each other via an amide bond.

13. The nucleic acid detection apparatus according to claim 9, wherein the effector protein and the particle are bound to each other via a linker.

14. The nucleic acid detection apparatus according to claim 13, wherein the linker contains a peptide formed of 6 or more and 11 or less consecutive histidine residues.

15. The nucleic acid detection apparatus according to claim 14, wherein the linker contains an antibody that binds to the peptide through an antigen-antibody reaction.

16. The nucleic acid detection apparatus according to claim 14, wherein the linker further contains a metal complex that binds to the peptide.

17. The nucleic acid detection apparatus according to claim 16, wherein the metal complex is a complex of: one of nitrilotriacetic acid or iminodiacetic acid; and a divalent nickel ion.

18. The nucleic acid detection apparatus according to claim 13, wherein the linker contains polyethylene glycol.

19. The nucleic acid detection apparatus according to claim 13, wherein the linker contains a complex of biotin and avidin.

20. The nucleic acid detection apparatus according to claim 9, wherein the particle has a particle diameter of 1 μm or more and 10 μm or less.

21. The nucleic acid detection apparatus according to claim 9,
wherein the distribution unit includes a recovery unit, and
wherein the recovery unit is configured to recover the target nucleic acid through use of a composite particle formed through binding between the effector protein bound to the particle and the crRNA.

22. The nucleic acid detection apparatus according to claim 1, wherein the individual independent separated compartments are liquid droplets.

23. The nucleic acid detection apparatus according to claim 1, wherein the individual independent separated compartments are wells.

24. The nucleic acid detection apparatus according to claim 1, wherein the individual independent separated compartments each have a volume of 0.1 fL or more and 1,000 fL or less.

25. The nucleic acid detection apparatus according to claim 1, wherein the individual independent separated compartments each have a volume of 0.5 fL or more and 400 fL or less.

26. The nucleic acid detection apparatus according to claim 1, wherein the identification unit is configured to identify each of the individual independent separated compartments having a fluorescence intensity exceeding the predetermined threshold value based on a ratio between a fluorescence intensity of a reference compartment and the fluorescence intensity of each of the individual independent separated compartments.

27. The nucleic acid detection apparatus according to claim 26, wherein the fluorescence intensity of the reference compartment is a fluorescence intensity acquired using a sample free of the target nucleic acid, and the detection reagent.

28. A method of detecting a nucleic acid comprising:
a distribution step of distributing a sample and a detection reagent to a plurality of individual independent separated compartments, the sample containing a target nucleic acid, and the detection reagent containing an effector protein, crRNA to be bound to the target nucleic acid, and a reporter molecule;
an activation step of activating the effector protein through binding of the crRNA to the target nucleic acid;
a fluorescence generation step of modifying the reporter molecule with the activated effector protein to generate fluorescence;
a fluorescence detection step of detecting the fluorescence; and
an identification step including determining, based on a detection result obtained in the fluorescence detection step, a fluorescence intensity of each of the individual independent separated compartments, and identifying each of the individual independent separated compartments having a fluorescence intensity exceeding a predetermined threshold value.

29. The method of detecting a nucleic acid according to claim 28,
wherein the effector protein is bound to a particle, and
wherein the distribution step comprises a recovery step of recovering the target nucleic acid through use of a composite particle formed through binding between the effector protein bound to the particle and the crRNA.

30. A non-transitory storage medium having stored thereon a program for causing a computer included in a nucleic acid detection apparatus to execute a method of detecting a nucleic acid so as to cause the nucleic acid detection apparatus to execute the method,
the method of detecting a nucleic acid comprising:
a distribution step of distributing a sample and a detection reagent to a plurality of individual independent separated compartments, the sample containing a target nucleic acid, and the detection reagent containing an effector protein, crRNA to be bound to the target nucleic acid, and a reporter molecule;
an activation step of activating the effector protein through binding of the crRNA to the target nucleic acid;
a fluorescence generation step of modifying the reporter molecule with the activated effector protein to generate fluorescence;
a fluorescence detection step of detecting the fluorescence; and
an identification step including determining, based on a detection result obtained in the fluorescence detection step, a fluorescence intensity of each of the individual independent separated compartments, and identifying each of the individual independent separated compartments having a fluorescence intensity exceeding a predetermined threshold value.
